(19) United States
(12) Patent Application Publication
(10) Pub. No.: US 2023/0332300 A1
(43) Pub. Date: Oct. 19, 2023

(54) ELECTROCHEMICAL SYSTEMS AND METHODS

(71) Applicants: Cam Med Inc., West Newton, MA (US); Massachusetts Institute of Technology, Cambridge, MA (US)

(72) Inventors: Chao Wang, Xiangxiang City (CN); Zhifei Ge, Cambridge, MA (US); Ju Li, Weston, MA (US)

(73) Assignees: Cam Med Inc., West Newton, MA (US); Massachusetts Institute of Technology, Cambridge, MA (US)

(21) Appl. No.: 18/027,275

(22) PCT Filed: Oct. 1, 2021
(86) PCT No.: PCT/US2021/053082

§ 371 Date: Mar. 20, 2023

Related U.S. Application Data
(60) Provisional application No. 63/086,647, filed on Oct. 2, 2020.

(51) Int. Cl. C25B 1/04

(52) U.S. Cl. CPC C25B 1/04

(57) ABSTRACT

Electrochemical systems and methods involving gas generation and/or consumption are generally described. In some aspects, an electrochemical system (e.g., an electrochemical cell) including a first electrode (e.g., an intercalation electrode) and a second electrode (e.g., for gas generation and/or consumption) is provided. Generation and/or consumption of gaseous species may be accomplished in some instances via application of voltages, and in some instances generated gas can deform components of the electrochemical system (e.g., compliant surfaces). The electrode materials may be chosen such that gas generation and/or consumption can be accomplished reversibly, controllably, and/or with relatively small energy input. Such properties may be useful in fluid pumping and/ or valving applications.

### RELATED APPLICATIONS

This application claims priority under 35 U.S.C. § 119(e) to U.S. Provisional Pat. Application Serial No. 63/086,647, filed Oct. 2, 2020, and entitled “Electrochemical Systems and Methods,” which is incorporated herein by reference in its entirety for all purposes.

### TECHNICAL FIELD

Electrochemical systems and methods involving gas generation and/or consumption are generally described.

### BACKGROUND

Some devices, such as pumps for fluids, involve actuators driven by electrical signals. Some types of actuators involve devices that undergo changes in volume and/or shape upon receiving an electrical signal. The change in volume and/or shape may cause movement of fluid.

Therefore, improved devices and methods for controlling volume and/or shape changes are desirable.

### SUMMARY

Electrochemical systems and methods involving gas generation and/or consumption are generally described. The subject matter of the present invention involves, in some cases, interrelated products, alternative solutions to a particular problem, and/or a plurality of different uses of one or more systems and/or articles.

In one aspect, electrochemical systems are provided. In some embodiments, an electrochemical system comprises a first electrode comprising an intercalation compound and a second electrode having an opposite polarity of the first electrode, wherein the electrochemical system is configured such that application of at least one magnitude of voltage across the first electrode and second electrode causes the intercalation compound in the first electrode to undergo an intercalation reaction and generates a gaseous species at the second electrode via a gas generation reaction, and wherein, under at least one condition, an equilibrium potential difference between the intercalation reaction and the gas generation reaction is less than or equal to 2 V.

In some embodiments, the electrochemical system comprises a first electrode comprising an intercalation compound and a second electrode having an opposite polarity of the first electrode, wherein the electrochemical system is configured such that application of a first magnitude of voltage across the first electrode and second electrode causes the intercalation compound in the first electrode to undergo an intercalation reaction and generates a gaseous species at the second electrode via a gas generation reaction, wherein the electrochemical system is configured such that application of a second magnitude of voltage across the first electrode and second electrode causes the intercalation compound in the first electrode to undergo a deintercalation reaction and consumes the gaseous species at the second electrode via a gas consumption reaction, and wherein the electrochemical system is configured such that when no electrical current is passed between the first electrode and the second electrode, the gas consumption reaction does not occur at the first electrode or the gas consumption reaction occurs at a rate of less than or equal to 5 mol%per day.

In some embodiments, the electrochemical system comprises a first electrode comprising an intercalation compound and a second electrode having an opposite polarity of the first electrode wherein the electrochemical system is configured such that application of at least one magnitude of voltage across the first electrode and second electrode causes the intercalation compound in the first electrode to undergo an intercalation reaction and generates a gaseous product at the second electrode via a gas generation reaction, and wherein the intercalation compound can undergo an intercalation reaction upon a first use of the electrochemical system.

In some embodiments, the electrochemical system comprises a chamber comprising a compliant surface at least partially enclosing an interior volume of the chamber, a first electrode exposed to the interior volume of the chamber and having a polarity, the first electrode comprising an electroactive compound comprising manganese or iron, and a second electrode exposed to the interior volume of the chamber and having an opposite polarity of the first electrode, wherein the electrochemical system is configured such that application of at least one magnitude of voltage across the first electrode and second electrode causes at least some of the manganese or iron to undergo a change in oxidation state and generates a gaseous species at the second electrode at a pressure sufficient to deform the compliant surface.

In another aspect, methods are provided. In some embodiments, a method comprises, in an electrochemical cell comprising a chamber comprising a compliant surface, a first electrode, and a second electrode in the chamber having an opposite polarity of the first electrode: applying a voltage having a magnitude of less than or equal to 3 V across the across the first electrode and second electrode such that a gaseous species is generated at the second electrode.

In some embodiments, a method comprises, in an electrochemical cell comprising a chamber comprising a compliant surface, a first electrode, and a second electrode in the chamber having an opposite polarity of the first electrode: applying a voltage across the first electrode and second electrode such that oxygen gas is generated at the second electrode and greater than or equal to 80 mole percent of a total amount of gas generated in the chamber during the applying step is oxygen gas, and deforming the compliant surface using the generated gas.

In some embodiments, a method comprises, in an electrochemical cell comprising a chamber, a first electrode in the chamber having a polarity, and a second electrode in the chamber having an opposite polarity of the first electrode: passing a first current through the first electrode and second electrode for a first period of time such that a gaseous species is generated; deforming the compliant surface using the generated gaseous species; passing a second current through the first electrode and second electrode for a second period of time such that a portion of the gaseous species is consumed; and determining an amount of the gaseous species consumed during the passing of the second current.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a cross-sectional schematic diagram of an electrochemical system comprising a chamber, a first electrode, a second electrode, and a compliant surface, according to some embodiments;

FIG. 2A shows a cross-sectional schematic diagram of an electrochemical system comprising a chamber, a first electrode, a second electrode, a compliant surface, and an electrolyte, according to some embodiments;

FIG. 2B shows a cross-sectional schematic diagram of an electrochemical system comprising a chamber, a first electrode, a second electrode, a compliant surface, an electrolyte, and generated gaseous species according to some embodiments;

FIG. 3A shows a cross-sectional schematic diagram of an electrochemical system comprising chamber, a first electrode, a second electrode, a compliant surface, an electrolyte, and a channel comprising fluid, according to some embodiments;

FIG. 3B shows a cross-sectional schematic diagram of an electrochemical system comprising a chamber, a first electrode, a second electrode, a compliant surface, an electrolyte, generated gaseous species, and a channel comprising fluid, according to some embodiments;

FIG. 4 shows a cross-sectional schematic diagram of an electrode comprising a substrate with a composite layer, according to some embodiments;

FIG. 5 shows a cross-sectional schematic diagram of an encapsulated electrochemical soft cell comprising a first electrode and a second electrode, according to some embodiments;

FIGS. 6A-6C shows images of porous electrodes, according to some embodiments;

FIG. 7 shows OER and ORR performance of a catalyst on a second electrode, according to some embodiments;

FIG. 8 shows a plot of OER and ORR performance of a catalyst during charge and discharge at 10 mA and 2 mA, according to some embodiments;

FIG. 9 shows a Pourbaix diagram of water splitting and an electrode potential of a first electrode, according to some embodiments;

FIGS. 10A-10C shows charge and discharge curves of first electrodes comprising (FIG. 10A) LiMn₂O₄ and (FIG. 10B) Li₄Mn₅O₁₂ discharged to -0.5 V, and (FIG. 10C) Li₄Mn₅O₁₂ discharged to -0.6 V versus Hg/HgO, according to some embodiments;

FIG. 11 shows a schematic of oxygen evolution and reduction volume over time according to some embodiments;

FIG. 12A shows charge and discharge profile of a full cell with a charge current of 2 mA and discharge current of 1 mA, according to some embodiments;

FIG. 12B shows the volume change of a full cell, according to some embodiments;

FIG. 13 shows cycling performance of Pt on stainless steel foam for the second electrode, and Li₄Mn₅O₁₂ for the first electrode, as a full cell in alkaline aqueous electrolyte, according to some embodiments;

FIG. 14 shows a perspective view schematic illustration of an electrochemical system connected to a pressure transducer, according to some embodiments;

FIG. 15 shows the measured pressure change during charge and discharge of a full cell, according to some embodiments;

FIG. 16 shows pressure measurement for 20 cycles of an electrochemical system with the full cell using a Pt catalyst for the second electrode formed by atomic layer deposition, according to some embodiments;

FIG. 17 shows changes in electrode potential with pH for certain reactions, according to some embodiments;

FIGS. 18A-18B show images of an assembled electrolytic chamber, with one port for electrolyte loading, according to some embodiments;

FIGS. 19A-19B show multistep current mode monitoring of an electrolysis voltage at 1, 5 and 10 mA with two pH levels, according to some embodiments;

FIGS. 20A-20B show typical electrolysis processes with 5×6 mm² 8.6 mg/cm² MnO₂ loaded electrodes and with pH 3 1 M phosphate buffer, according to some embodiments;

FIGS. 21A-21D show electrolysis with 5×6 mm² 15 mg/cm² loaded MnO₂ electrodes with pH 3 1 M phosphate buffer, according some embodiments;

FIG. 22 shows 150 µL delivery at 2 mA electrolysis with 15 mg/cm² MnO₂ electrode, according to some embodiments;

FIG. 23 shows results for a ~ 5×6 mm 16 mg/cm² MnO₂ electrode soaked overnight in regular 1 M pH 3 buffer and 1 M pH 3 buffer with saturated Mn²⁺, according to some embodiments;

FIG. 24 shows a direct comparison of single-sided and double sided MnO₂ electrodes, according to some embodiments; and

FIG. 25 shows a comparison of the performance of different single sided MnO₂ coatings in closed chambers with 5×6 mm² with 2 mA electrolysis, according to some embodiments.

### DETAILED DESCRIPTION

Electrochemical systems and methods involving gas generation and/or consumption are generally described. In some aspects, an electrochemical system (e.g., an electrochemical cell) including a first electrode (e.g., an intercalation electrode) and a second electrode (e.g., for gas generation and/or consumption) is provided. Generation and/or consumption of gaseous species may be accomplished in some instances via application of voltages and passage of electrical current, and in some instances generated gaseous species can deform components of the electrochemical system (e.g., compliant surfaces). The electrode materials may be chosen such that gas generation and/or consumption can be accomplished reversibly, controllably, and/or with relatively small energy input. Such properties may be useful in fluid pumping and/or valving applications.

Certain mechanical systems involving fluid control, such as pumping and valving, employ actuators. Some actuators involve volume and/or shape changes due to gas generation (e.g., via electrolytic gas generation). Certain existing systems use water electrolysis into hydrogen (H₂) and oxygen (O₂) molecules as a mechanism for actuation. However, it has been realized in the context of this disclosure that hydrogen is much more permeable through commonly used actuator materials (e.g., membranes made of plastics and other materials) than is oxygen, and it can be difficult to keep H₂ inside a cell over time periods of days to weeks. While hydrogen leakage can in some instances be mitigated by better barrier materials, there still may be some spontaneous volume shrinkage due to hydrogen permeation, which may cause problems in certain applications, such as drug delivery applications.

It has also been realized that during water electrolysis into hydrogen and oxygen, only gas generation occurs in an electrically controllable manner, and one cannot reduce gas volume in the actuator in an electrically controllable fashion. Certain existing approaches use platinum-group metals to catalyze the electrolysis of water, but the same catalyst can also facilitate the recombination of hydrogen and oxygen into water even without an energy input. Such recombination is not electrically controllable, especially when no good barrier material can be found to stop H₂ from leaving the proximity of the hydrogen-forming electrode region and approaching the oxygen-forming electrode. In this context, controllable means being able to regulate the rate of gas generation/consumption by metering the external electrical current of the electrochemical cell. The recombination of hydrogen and oxygen molecules to produce water in an encapsulated cell is thermodynamically spontaneous and may not generate an electrical current through the external circuit. Aspects of the present disclosure are directed to addressing these potential challenges in electrolytic gas-driven actuators by employing methods and electrode materials that in some instances produce relatively little hydrogen, and in some instances allow for controllable, reversible gas generation/consumption with relatively low energy expenditure. For example, in some embodiments, a volume of gas in an electrochemical system may change only by application of electrical current in forward or reverse directions.

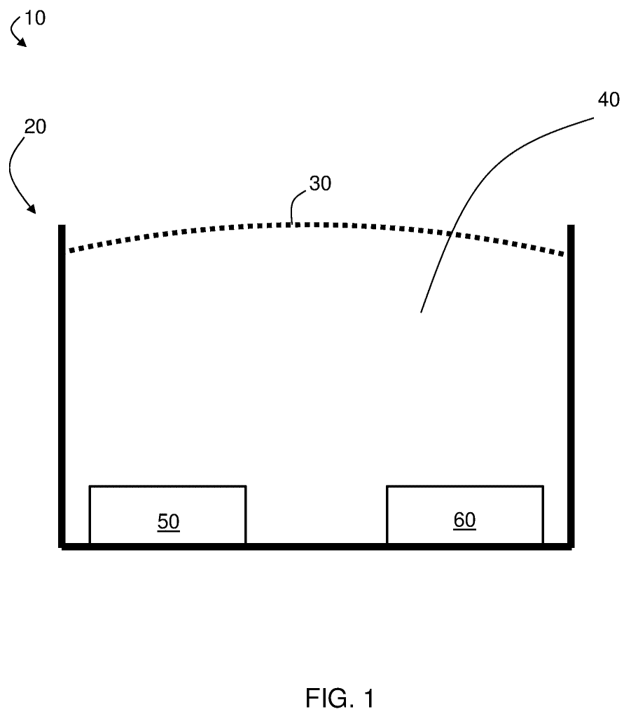

In one aspect, electrochemical systems are provided. In some embodiments, the electrochemical systems are configured to be used as actuators (e.g., for causing the movement of fluids such as in pumping and valving applications). FIG. 1 shows a cross-sectional schematic diagram of an electrochemical system 10, according to some embodiments. In the embodiment shown in FIG. 1, electrochemical system 10 comprises a chamber 20 and a compliant surface 30 at least partially enclosing an interior volume 40, in which a first electrode 50 and a second electrode 60 reside. It should be understood that any of a variety of suitable configurations for electrochemical systems capable of the chemistries described herein are possible, and the configuration shown in FIG. 1 with a chamber and compliant surface is for illustrative purposes and not meant to be limiting. For example, in some embodiments the electrochemical system does not comprise a compliant surface, and instead comprises one or more other elements capable of accomplishing actuation upon gas generation/consumption, such as solid surfaces such as in pistons.

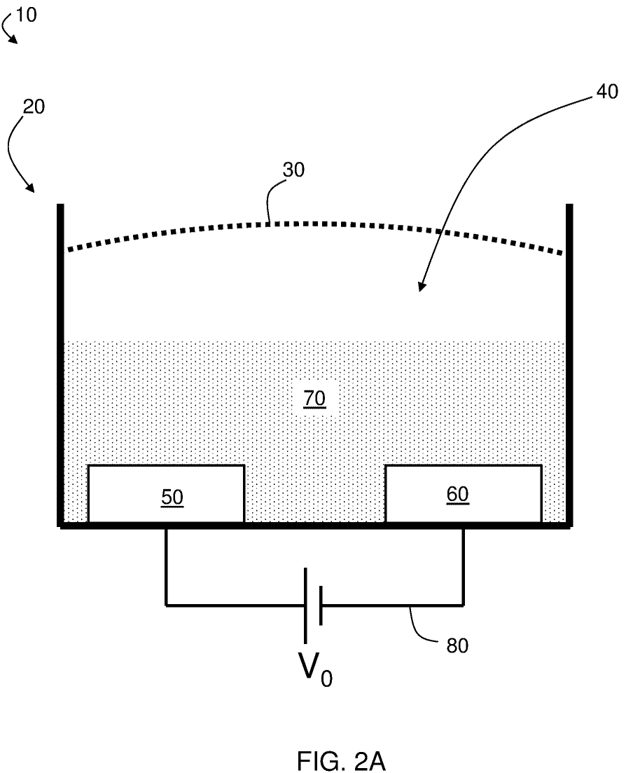

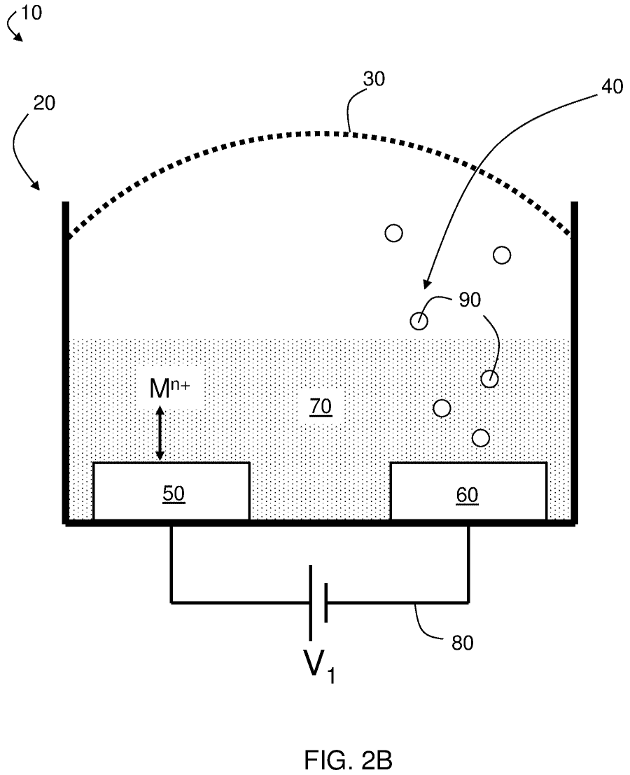

The electrochemical system may be configured to be an electrochemical cell. For example, in some embodiments, an electrolyte is present and in contact with one or both of the first electrode and the second electrode. Referring now to FIG. 2A, electrochemical system 10 may comprise an electrolyte 70 within chamber 20, according to some embodiments. In some embodiments, application of at least one magnitude of voltage across the first electrode and second electrode can induce an electrochemical reaction comprising two or more redox half reactions. For example, referring again to FIG. 2A, application of a voltage across first electrode 50 and second electrode 60 via external electrical circuit 80 may initiate an electrochemical reaction. In some instances, when the electrochemical system is at an open circuit (as shown as V₀ in FIG. 1A), little to no electrochemical reactivity is observable. According to some embodiments, reactivities and structural properties of the materials of the first electrode (e.g., an intercalation electrode) and second electrode (e.g., a gas generation/consumption electrode) may promote gas generation and consumption in a manner suitable for actuation (e.g., for fluid pumping and/or valving purposes, such as for drug infusion for human or animal patients). For example, gas generation may occur with relatively little or no hydrogen generation.

Voltage may be applied across electrodes using any of a variety of devices and techniques known in the art, such as via a power source (e.g., one or more batteries), via a potentiostat, and the like, depending on the system configuration. It should also be understood that in some embodiments a voltage may be applied using a preselected voltage (e.g., based on a battery voltage or potentiostat setting). However, in some embodiments, the applied voltage is not pre-selected. For example, a desired electrical current (or current density) may be selected, and the system may apply a voltage necessary to achieve such a current.

Gas generation may occur in the electrochemical system. Referring to FIG. 2B, application of a voltage V₁ across first electrode 50 and second electrode 60 via electrical circuit 80 may induce formation of a gaseous species 90 within interior volume 40. While gaseous species 90 is shown as observable bubbles 90 in FIG. 2B, it should be understood that gaseous species may occupy volume without forming bubbles in electrolyte 70. In other words, the gaseous species maybe be in the form of dissolved molecules or in the form of attached or detached gas bubbles. The gaseous species generated (e.g., at the first electrode) may cause a shape and/or volume change of a component of the electrochemical system (e.g., a change in volume of the chamber). For example, in some embodiments involving compliant surfaces, operation of the electrochemical system causes deformation of a compliant surface. For example, gas generated in the system (e.g., oxygen gas generated at the second electrode) may cause a pressure sufficient to deform a compliant surface. In FIG. 2B, for example, gaseous species 90 may cause compliant surface 30 to deform outwardly from chamber 20.

As mentioned above, the system may be configured to reduce or eliminate hydrogen gas generation (e.g., during electrolysis of an electrolyte). One way in which the system can be configured to limit hydrogen generation is via the use of an intercalation electrode as the first electrode, as described in more detail below. In some embodiments, the electrochemical system is configured such that application of at least one magnitude of voltage across the first electrode and second electrode (1) causes the intercalation compound in the first electrode to undergo an intercalation reaction, and (2) generates a gaseous species at the second electrode via a gas generation reaction. For example, referring to FIG. 2B again, as current is passed through the circuit defined by first electrode 50, second electrode 60, electrolyte 70, and electrical circuit 80 during application of voltage V₁, gas generation may occur at second electrode 60 while an ion Mⁿ⁺ undergoes intercalation or deintercalation with an intercalation compound of first electrode 50. In some embodiments, rather than an intercalation reaction, an ion (e.g., ion Mⁿ⁺) of the first electrode (e.g., first electrode 50) undergoes a change in oxidation state (e.g., to form Mⁿ⁺¹, Mⁿ⁺², Mⁿ⁻¹, Mⁿ⁻², etc.). As a non-limiting example, the first electrode may comprise an electroactive compound comprising manganese or iron. In some such embodiments, the electrochemical system is configured such that application of at least one magnitude of voltage across the first electrode and second electrode (1) causes at least some of the manganese or iron to undergo a change in oxidation state and (2) generates a gaseous species at the second electrode at a pressure sufficient to deform a compliant surface. It has been realized in the context of the present disclosure that configuring a system to have a first electrode undergo intercalation/deintercalation and/or oxidation/reduction reactions can result in a complete electrochemical reaction where a desirable gaseous species (e.g., oxygen) is generated at a second electrode while generation of an undesirable gaseous species (e.g., hydrogen) is limited or prevented altogether at the first electrode.

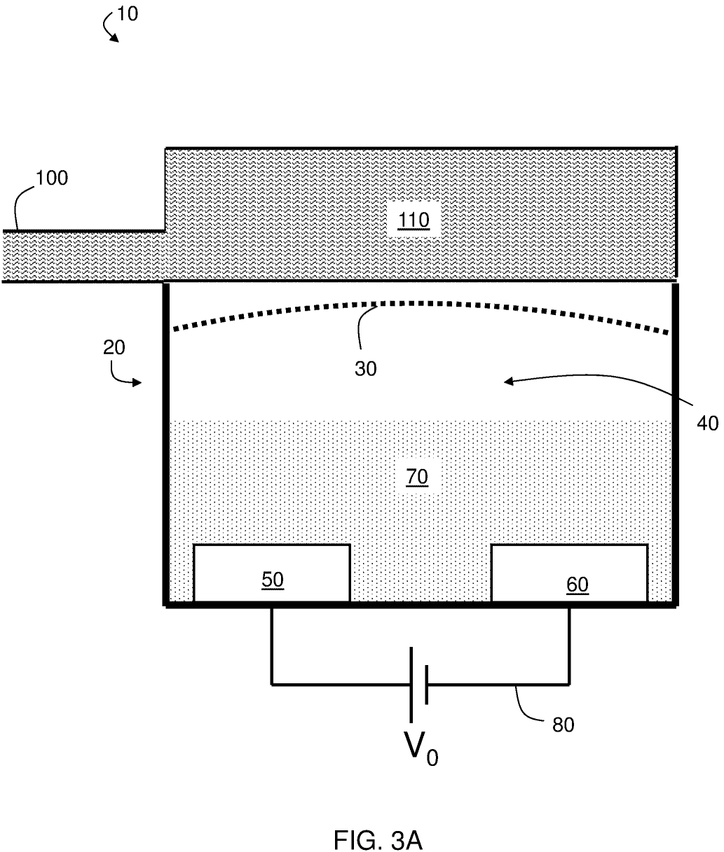

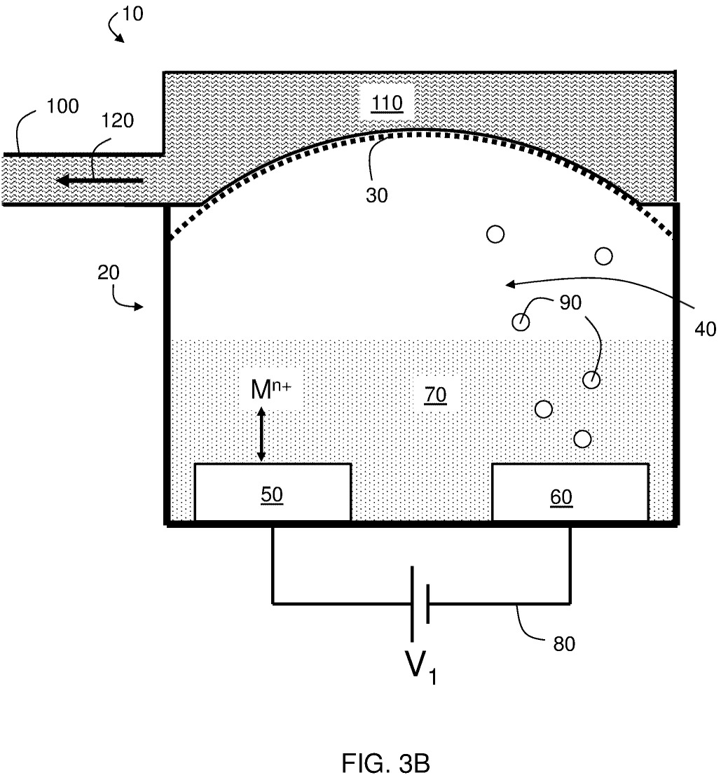

In some embodiments, deformation of a compliant surface (e.g., from pressure from a generated gaseous species) causes a fluid to flow at least partially through a channel. The channel may be part of, for example, a fluidic device configured to transport and/or dispel fluid. For example, the fluid may be a liquid. In such a way, the methods and systems described herein may be configured to initiate the flow of fluids (e.g., liquids) for any of a variety of applications, such as pumping fluids for medical applications (e.g., delivery of therapeutics in liquid form). Referring to FIGS. 3A-3B, electrochemical system 10 may further comprise a channel 100 comprising a fluid (e.g., liquid) 110, according to some embodiments. In some embodiments, electrochemical system 10 is configured such that in the absence of sufficient pressure from a gaseous species compliant surface 30 is in a first position/shape (e.g., when electrochemical system 10 is at open-circuit V₀, FIG. 3A), but in the presence of sufficient pressure from generated gaseous species 90 compliant surface 30 deforms to a second position/shape that displaces at least a portion of fluid 110, causing flow of some or all of fluid 110 through channel 100 (e.g., in a direction indicated by arrow 120; FIG. 3B). The channel may be in fluid communication with the compliant surface. That is, fluid in the channel may be able to directly contact the compliant surface. For example, the compliant surface may form a part of the channel. However, in some embodiments, one or more intervening structures (e.g., layers such as other compliant surfaces) are between the channel and the compliant surface.

In some embodiments, the electrochemical system is configured such that application of a first magnitude of voltage across the first electrode and second electrode (1) causes the intercalation compound in the first electrode to undergo an intercalation reaction, and (2) generates a gaseous species at the second electrode via a gas generation reaction, and the electrochemical system is configured such that application of a second magnitude of voltage across the first electrode and second electrode (1) causes the intercalation compound in the first electrode to undergo a deintercalation reaction, and (2) consumes the gaseous species at the second electrode via a gas consumption reaction. For example, application of a first voltage may cause generation of oxygen gas at the second electrode (e.g., from water electrolysis), and application of a second, different voltage may cause generation of H₂O at the second electrode via the consumption of oxygen gas (e.g., via the ORR reaction), while the first electrode undergoes ion intercalation and deintercalation processes.

In some embodiments, the electrochemical system is configured to generate gaseous species reversibly. That is, the system may be configured to generate gaseous species under a first configuration (e.g., during application of a first voltage) and consume a relatively high amount of the generated gaseous species under a second configuration (e.g., during application of a second, different voltage). In some embodiments, a reversible system can undergo an electrochemical reaction that consumes at least 30 mole percent (mol%), at least 40 mol%, at least 50 mol%, at least 60 mol%, at least 70 mol%, at least 75 mol%, at least 80 mol%, at least 85 mol%, at least 90 mol%, at least 95 mol%, at least 98 mol%, at least 99 mol%, at least 99.9 mol%, or 100 mol% of generated gas. In some embodiments, a reversible system can undergo an electrochemical reaction that consumes less than or equal to 100 mol%, less than or equal to 99.9 mol%, less than or equal to 99 mol%, less than or equal to 98 mol%, less than or equal to 98 mol%, less than or equal to 95 mol%, less than or equal to 90 mol%, less than or equal to 85 mol%, less than or equal to 80 mol%, less than or equal to 70 mol%, less than or equal to 60 mol%, less than or equal to 50 mol%, less than or equal to 40 mol%, less than or equal to 30%, or less of the generated gas. Combinations of these ranges (e.g., at least 30 mol% and less than or equal to 100 mol%) are possible.

In some embodiments, the electrochemical system comprises a first electrode. In some embodiments, the first electrode is not a gas generation/gas consumption electrode. In some embodiments, the first electrode comprises an electroactive species. An electroactive species generally refers to a species able to undergo an electrochemical reaction. In some embodiments, the first electrode comprises an intercalation compound. An intercalation compound generally refers to a compound capable of reversibly inserting an ion at lattice sites and/or interstitial sites of the compound. For example, during a first electrochemical process, an intercalation compound may undergo an intercalation reaction by intercalating an ion (e.g., from a neighboring medium such as an adjacent electrolyte) such that the ion is inserted at a lattice site and/or an interstitial site of the intercalation compound. Then, during a second electrochemical process, the intercalation compound may undergo a deintercalation reaction by deintercalating the ion such that the ion is released (e.g., into a neighboring medium such as an adjacent electrolyte). As such, the intercalation compound may be able to reversibly undergo the intercalation and the deintercalation reaction. Any of a variety of intercalation compounds may be suitable for the first electrode. In some embodiments, the intercalation compound is a metal ion/proton intercalation compound. That is, the intercalation compound may be capable of intercalating and deintercalating metal ions and/or protons. Examples of suitable metal ions include, but are not limited to alkali ions (e.g., lithium ions, sodium ions, potassium ions), alkaline earth metal ions (e.g., magnesium ions, calcium ions, strontium ions), protons (H⁺), and hydroxide ions (OH⁻). In some embodiments, the intercalation compound comprises a material having a relatively high mixed ionic/electronic conductivity. In some embodiments, the intercalation compound comprises a lithium ion intercalation compound. In some embodiments, the intercalation compound comprises a transition metal oxide. For example, the intercalation compound may comprise a manganese oxide (e.g., a lithium manganese oxide), a cobalt oxide, an iron oxide, a nickel oxide, or oxides comprising combinations thereof. In some embodiments, the intercalation compound comprises LiMnO₂, LiMn₂O₄, and/or Li₄Mn₅O₁₂. Other stoichiometries are also possible. As another example, Prussian white analogues (KₓM_{y}[Fe(CN)₆]_{z} (M ═ Fe, Co, Ni, Cu)) and Prussian blue (KFe[Fe(CN)_{6]}), etc. could also be used for the first electrode. In some embodiments, the intercalation compound comprises a transition metal oxyanion. One non-limiting example of a transition metal oxyanion intercalation compound is a compound comprising an iron phosphate (e.g., lithium iron phosphate, LiFePO₄). In some embodiments, a surface layer at least partially coats the first electrode. The surface layer may be configured to decrease side reactions. In some embodiments, a surface layer (e.g., coating) decreases side reactions. Examples of potentially suitable surface coatings include, but are not limited to, polymers (e.g., Nafion™, PTFE, PEDOT-PSS, PANI, PI, etc.) and/or ceramics (Al₂O₃, TiO₂, etc.)

In some embodiments, the intercalation compound can undergo an intercalation reaction upon a first use (e.g., first voltage application) of the electrochemical system. For example, the intercalation compound of an ion (e.g., a lithium ion) may comprise vacant sites for that ion (e.g., vacant sites into which Li⁺ may be inserted). It has been realized that for embodiments in which the system is configured to generate gas at the second electrode while intercalating an ion at an intercalation compound of the first electrode, having vacant sites present in the intercalation compound prior to first use can facilitate gas generation upon initial voltage application, rather than needing to undergo a first deintercalation step in the device prior to gas generation. Electrode materials like MnO₂, LiMn₂O₄, Li₄Mn₅O₁₂, FePO₄, V₂O₅, etc. can have vacant atomic sites and thus can be used in such electrochemical systems in which intercalation is desired upon initial voltage application. In some embodiments, the intercalation compound can be chemically synthesized such that vacant sites for the ion to be intercalated are present.

Alternatively, one can achieve an intercalation compound comprising vacant sites not only directly in chemical synthesis, but also by electrochemically deintercalating (e.g., delithiating) materials like LiFePO₄ that may be synthesized with a full complement of intercalated ions. Referring to the LiFePO₄ reaction, the following electrochemical delithiation step may be performed.

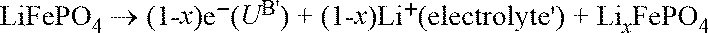

This electrochemical reaction may occur as a preparatory step in a separate electrochemical system, in a liquid electrolyte that may differ from that of an electrolyte used in the gas generation/consumption electrochemical system. This electrochemical reaction may be a pre-processing step, after which the different liquid electrolyte is washed away from the electrode. For example, materials like LiMn₂O₄, Li₄Mn₅O₁₂, LiFePO₄ can be slurry coated on the current collector and charged (delithiated) first by the above-mentioned electrochemical reaction, washed, and then an entire electrode loaded with LiₓMn₂O₄, LiₓMn₅O₁₂, LiₓFePO₄ may be harvested, (e.g., divided by cutting) and assembled into the completed electrochemical system described herein.

The intercalation compound may be able to be cycled (e.g., undergo reversible intercalation and deintercalation reactions) in alkaline electrolytes (e.g., having a pH of greater than or equal to 9). In some embodiments, the first electrolyte comprises one or more additives. Examples of additives that may be employed in some embodiments include, but are not limited to, lithium salts, such as Li₂SO₄, LiNO₃, and the like. The additives may decrease an equilibrium potential of a hydrogen evolution reaction compared to that of an otherwise identical electrolyte lacking the one or more additives. Inclusion of such additives may therefore, in some embodiments, suppress deleterious hydrogen gas production.

In some embodiments, the first electrode comprises an electroactive compound comprising manganese or iron. For example, the first electrode may comprise a manganese oxide (e.g., manganese (IV) oxide, MnO₂). Upon application of a voltage, a reduction reaction may occur in which an oxidation state of the electroactive compound changes. For example, Mn(IV) in MnO₂ may be reduced to Mn(II). In some embodiments, such a reaction may result in release of Mn²⁺ (e.g., into neighboring electrolyte). In some embodiments, such a reduction reaction at the first electrode may occur simultaneously with a gas generation reaction at the second electrode (e.g., oxygen gas generation). In some embodiments, the electroactive compound (e.g., intercalation compound, manganese or iron compound) is air-stable.

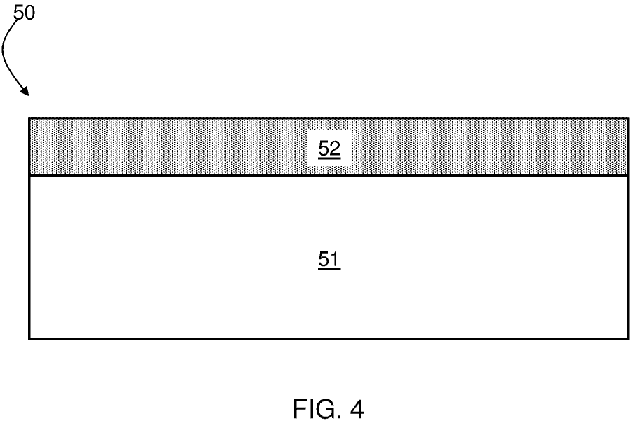

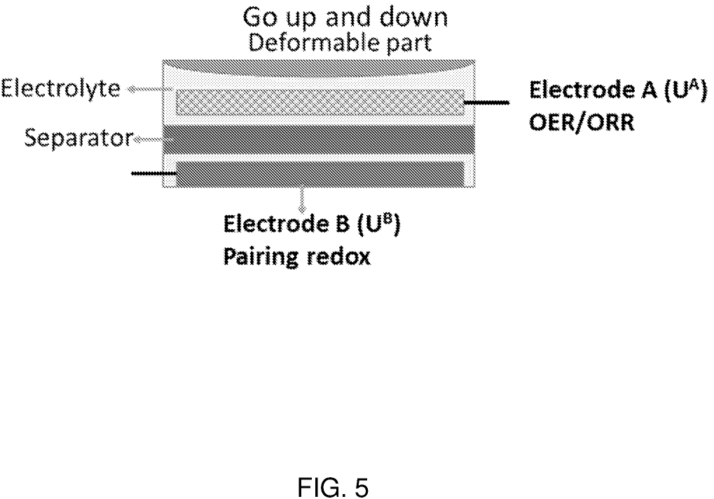

In some embodiments, the first electrode (e.g., electrode B in FIG. 5) comprises an electroactive compound (e.g., intercalation compound) that is part of a composite (e.g., a composite layer). In some embodiments, the first electrode comprises a substate and the electroactive compound on at least a portion (e.g., some or all) of the substrate. Referring to FIG. 4, first electrode 50 may comprise a substrate 51 with a composite layer 52 (e.g., comprising an electroactive compound) on substrate 51, according to some embodiments. The substrate may comprise an electrically conductive material (e.g., an electrically conductive solid) and serve as a current collector during electrochemical reactions (e.g., facilitating flow of electrons to an external circuit). In some embodiments, a transition metal and/or transition metal alloy is employed. For example, an iron alloy be used. One suitable type of iron alloy is stainless steel (e.g., 304, 316, 316L type stainless steel). In some embodiments, a non-platinum substrate comprises titanium (e.g., titanium metal) and/or a titanium alloy.

The first electrode can be manufactured using any of a variety of techniques. For example, the first electrode can be manufactured by a slurry casting process. In some embodiments, electroactive materials (MnO₂, LiMn₂O₄, Li₄Mn₅O₁₂, FePO₄, V₂O₅, LiₓMn₂O₄, LiₓMn₅O₁₂, LiₓFePO₄, etc.), conductive agents such as carbon black or carbon nanotubes, and binders such as polyvinylidene difluoride (PVDF), polytetrafluoroethylene (PTFE), and/or polyimide (PI) are mixed to form a uniform slurry first. Then the slurry may cast on a substrate (e.g., a stainless steel mesh current collector). The current collector should, in some embodiments, be stable in the electrolyte and during oxidation and reduction processes. The use of metallic mesh may prevent spallation of the slurry from the current collector.

In some embodiments, the electrochemical system comprises a second electrode (e.g., electrode A in FIG. 5). In some embodiments, the second electrode is a gas generation/gas consumption electrode. In some embodiments, the second electrode has an opposite polarity of the first electrode. That is, when the first electrode and second electrode are part of an electrochemical cell undergoing an electrochemical reaction involving an oxidation half reaction and a reduction half reaction, the oxidation half reaction may occur at the first electrode and the reduction half reaction may occur at the second electrode, or vice versa.

As mentioned above, application of at least one magnitude of voltage may result in generation of a gaseous species at the second electrode. In some embodiments, the gaseous species is oxygen gas (O₂). The oxygen gas may be generated via the oxidation of water (H₂O), in a reaction referred to herein as the oxygen evolution reaction (OER). In some embodiments, application of at least one magnitude of voltage may result in consumption of a gaseous species (e.g., at the second electrode). For example, in some embodiments where oxygen gas is generated, application of a voltage may result in reduction of oxygen gas to form a non-gaseous product (e.g., hydrogen peroxide or water). In some embodiments, oxygen gas is reduced at the second electrode to produce water, in a reaction referred to herein as the oxygen reduction reaction (ORR). In some embodiments, the second electrode comprises one or more catalysts for an oxygen evolution reaction and/or an oxygen reduction reaction. One of ordinary skill in the art, with the benefit of this disclosure would know of suitable catalysts for the OER and ORR reactions, and non-limiting examples are provided below. In some embodiments, the second electrode comprises a substrate. The substrate may have the same or similar composition to those described above in the context of the first electrode, and may serve as a current collector. In some embodiments, but not necessarily all embodiments, for example, the second electrode comprises a non-platinum substrate. In some embodiments, the substrate of the second electrode is at least partially coated with an oxygen-reduction catalyst (and/or an oxygen-evolution catalyst). In some embodiments, the first electrode and/or the second electrode is at least partially coated with a polymer electrolyte.

In some embodiments, the second electrode is configured to generate bubbles of the gaseous species. It has been discovered that depending on electrode configuration and reaction conditions, different types of bubbles may be formed. Some bubbles may detach from the second electrode (and move away from the second electrode into a bulk of an electrolyte). However, some bubbles may remain attached to the second electrode. It has been realized that both detached bubbles and attached bubbles may contribute to a volume of gas in the electrochemical system, and both may therefore contribute to a gas pressure experienced, for example, by a deformable compliant surface. However, it has been realized that in some embodiments where reversible gas generation and consumption is desired, promoting formation of attached bubbles versus detached bubbles can facilitate reversible reactivity. It is believed that there is a lower kinetic barrier to consuming gaseous species in the form of attached bubbles compared to detached bubbles. Any of a variety of techniques may be employed to promote attached bubbles of gas compared to detached bubbles. For example, a porosity of the second electrode may be selected to promote attached bubbles. In some embodiments, for example, the second electrode comprises a three dimensional porous current collector. Such a three dimensional porous current collector (e.g., comprising an electrically conductive solid material) may have a pore structure that promotes formation and retention of attached gas bubbles. In some embodiments, the three dimensional porous current collector is in the form of a stainless steel foam or folded mesh film (e.g., 316, 316L, and/or 654 stainless steel). In some embodiments, a surface of the porous framework can be coated with a dense layer of a metal and/or metal alloy (e.g., Ti) or other conductive and stable materials that can improve stability in a strong alkaline and oxidative environment. In some embodiments, the second electrode is at least partially coated with a materials that can improve the wettability for an interface between the current collector and the generated gaseous species (e.g., oxygen gas). Such a coating for improving wettability could include materials comprising a polymer and/or metal oxide (e.g., Nafion, PEDOT-PSS, PDMS, PANI, Al₂O₃, TiO₂, etc.).

In some embodiments, the three dimensional porous current collector comprises open-channel pores with largest cross-sectional dimensions ranging from 10 nm to 1 mm. For example, the pores may have largest cross-sectional dimensions that are greater than or equal to 10 nm, greater than or equal to 50 nm, greater than or equal to 100 nm, greater than or equal to 500 nm, greater than or equal to 1 micrometer, greater than or equal to 5 micrometers, greater than or equal to 10 micrometers, greater than or equal to 50 micrometers, and/or up to 100 micrometers, up to 500 micrometers, up to 1 mm, or more. The porosity of a material in this context, including largest cross-sectional dimensions of pores, can be determined using mercury porosimetry experiments.

Additionally, it has been realized that for a given total current and rate of OER or ORR, a porous structure of the first electrode can decrease the overpotential by increasing the electrochemical surface area, which may lower the electrical energy consumption per volume change. A porous substrate (e.g., a porous stainless-steel foam (e.g., 304, 316, 316 L type stainless steel or a titanium foam) may be suitable for a variety of conditions, including potentially harsh environments.

In some embodiments, a second electrode comprises a noble metal catalyst on the porous substrate. In some instances, noble metal catalysts can contribute to better OER and ORR catalyst performance at the second electrode. Noble metals such as Platinum, Palladium, Iridium, Ruthenium, and their combinations, or their oxides (including Tantalum, Niobium oxides), could work in some embodiments. For example, Pt coated porous titanium, Pt coated porous stainless steel, mixed metal oxide coated titanium foam, Pt-mixed metal oxide coated titanium mesh, Iridium coated titanium foam, etc. can be suitable choices for the second electrode. In some embodiments in which a Pt catalyst is present, the catalyst comprises Pt and/or Pt alloy nanoparticles. Doping by nitrogen, mercury, etc. may also useful as it can suppress hydrogen evolution reaction (HER), which can be a parasitic side reaction that can happen at certain voltages. The coating method can be varied. Electrochemical plating, electroless plating, high-temperature sintering, sputtering, chemical vapor deposition, or atomic layer deposition may be employed.

In a particular implementation, Pt is coated on 316L stainless steel foam by an electrochemical plating process. For example, H₂PtCl₆.6H₂O may be used as the Pt precursor in an H₂SO₄ solution. Pt metallic catalysts may be coated using cyclic voltammetry, or pulse current/voltage methods.

Alternatively, for electroless deposition, a thin layer of copper or nickel or titanium/nickel may first be coated on a substrate (e.g., stainless-steel foam). A replacement reaction with H₂PtCI₆ or a different noble metal salt may follow, to deposit metallic Pt. Atomic layer deposition (ALD), sputtering, and/or high-temperature sintering methods can also be efficient for Pt loading of a second electrode. Especially, ALD can be controlled to deposit 1-2 nm thick Pt, which can decrease the materials cost.

In some embodiments, the second electrode is configured such that a starting potential of the OER is less than or equal to (less positive than) 0.6 V and ORR is greater than or equal to (more positive than) 0 V versus a Hg/HgO standard electrode. Such potentials may be achievable by using catalysts to reduce overpotentials for the OER and/or ORR reactions.

It has been realized herein that systems and methods involving an intercalation reaction at a first electrode and a gas generation reaction at a second electrode can be configured such that a relatively low energy input is required for gas generation. As one example, in some embodiments, an intercalation compound of the first electrode may be such that an equilibrium potential difference between the intercalation compound and the gas generation reaction is relatively small. Such a small equilibrium potential difference (open circuit potential difference), which can be determined based on known redox half reaction reduction potentials and known reaction conditions (e.g., temperature, pH) may allow for relatively small applied voltages to be applied. Having a relatively small equilibrium potential difference between the intercalation material of the first electrode (e.g., a lithium transition metal oxide) and a gas generation reaction (e.g., OER) stands in fundamental contrast to other, different devices that may employ intercalation materials and gas generation/consumption materials, such as batteries. With batteries, relatively high equilibrium potential differences are desired in order to achieve high battery voltages/energy densities. Example 1 below describes exemplary calculations of equilibrium potential differences and design criteria, according to some embodiments.

In some embodiments, under at least one condition (e.g., temperature, pH), an equilibrium potential difference between the intercalation reaction and the gas generation reaction is less than or equal to 2 V, less than or equal to 1.8 V, less than or equal to 1.6 V, less than or equal to 1.5 V, less than or equal to 1.4 V, less than or equal to 1.3 V, less than or equal to 1.2 V, less than or equal to 1.1 V, less than or equal to 1 V, less than or equal to 0.9 V, less than or equal to 0.8 V, less than or equal to 0.7 V, less than or equal to 0.6 V, less than or equal to 0.5 V, less than or equal to 0.4 V, less than or equal to 0.3 V, less than or equal to 0.2 V, less than or equal to 0.1 V, less than or equal to 0.0 V, or less. In some embodiments, under at least one condition (e.g., temperature, pH), an equilibrium potential difference between the intercalation reaction and the gas generation reaction is greater than or equal to -1 V, greater than or equal to -0.9 V, greater than or equal to -0.8 V, greater than or equal to -0.7 V, greater than or equal to -0.6 V, greater than or equal to -0.5 V, greater than or equal to -0.4 V, greater than or equal to -0.3 V, greater than or equal to -0.2 V, greater than or equal to -0.1 V, greater than or equal to 0.0 V, greater than or equal to 0.05 V, greater than or equal to 0.1 V, greater than or equal to 0.15 V, greater than or equal to 0.12 V, greater than or equal to 0.3 V, greater than or equal to 0.4 V, greater than or equal to 0.5 V, or greater. Combinations of these ranges (e.g., greater than or equal to -1 V and less than or equal to 2 V, greater than or equal to -1 V and less than or equal to 1 V) are possible. Such equilibrium potentials may depend, for example, on the pH of the reaction conditions. In some embodiments, the equilibrium potential difference is within any of the above-mentioned ranges in an aqueous solution having a pH from 0 to 16, 3 to 16, 6 to 16, or 6 to 14.

In some embodiments, the electrochemical system comprises an electrolyte. The electrolyte may be in contact with the first electrode and/or the second electrode. The electrolyte may be a liquid electrolyte (e.g., an electrolyte solution) or a solid electrolyte, depending on the system configuration. In some embodiments in which a system comprises a chamber (e.g., at least partially enclosed by a compliant surface), an interior volume of the chamber can be at least partially filled with an electrolyte solution. In some embodiments, the electrolyte solution is an aqueous electrolyte solution (e.g., a solution comprising water in an amount of greater than or equal to 10 weight percent (wt%), greater than or equal to 50 wt%, greater than or equal to 75 wt%, greater than or equal to 90 wt%, greater than or equal to 90 wt%, greater than or equal to 95 wt%, greater than or equal to 98 wt%, greater than or equal to 99 wt%, greater than or equal to 99.9 wt%, or higher). The electrolyte solution may provide a reactant for an electrochemical reaction in the system. For example, water from an aqueous electrolyte solution may be oxidized to form oxygen gas during a gas generation solution. In some embodiments, the electrolyte comprises one or more additives. For example, the electrolyte may comprise a supporting electrolyte comprising dissolved ions (e.g., from a salt). In some embodiments, the electrolyte comprises one or more buffers (e.g., for maintaining a relatively stable pH during reactions involving protons and/or hydroxide ions). In some embodiments, the electrolyte solution comprises one or more dissolved salts comprising an ion that can be intercalated into and deintercalated from the intercalation compound of the first electrode. For example, in some embodiments where the first electrode comprises a lithium intercalation compound, the electrolyte comprises dissolved forms of one or more of the following salts: LiOH, Li₂SO₄, Li₂ClO₄, LiTFSI, LiFSI, in the concentration range of from 0.1 M to 10 M.

In some embodiments, the pH of an electrolyte solution can contribute to advantageous reactivity. For example, in some embodiments where it is desirable for a relatively fast gas generation reaction or a relatively low equilibrium potential difference between an intercalation reaction and a gas generation reaction (e.g., OER), an alkaline pH (e.g., greater than 7) may be employed. In some embodiments, the pH of an electrolyte solution is greater than or equal to 0, greater than or equal to 1, greater than or equal to 2, greater than or equal to 3, greater than or equal to 4, greater than or equal to 5, greater than or equal to 6, greater than or equal to 7, greater than or equal to 8, greater than or equal to 9, or greater. In some embodiments, the pH of an electrolyte solution is less than or equal to 16, less than or equal to 15, less than or equal to 14, less than or equal to 13, less than or equal to 12, less than or equal to 11, less than or equal to 10, less than or equal to 9, less than or equal to 8, less than or equal to 7, less than or equal to 6, less than or equal to 5, less than or equal to 4, or less. Combinations of these ranges (e.g., greater than or equal to 0 and less than or equal to 16, greater than or equal to 0 and less than or equal to 4, greater than or equal to 6 and less than or equal to 16, greater than or equal to 6 and less than or equal to 14, greater than or equal to 7 and less than or equal to 14, or greater than or equal to 9 and less than or equal to 14) are possible.

As mentioned above, the electrochemical system may comprise a chamber. The chamber may have an interior volume. The chamber may have a shape such that in the absence of other components such as electrodes and electrolyte, the interior volume is not occupied by any solid. For example, the chamber may comprise a bottom portion and side portions and optionally a top portion defining an interior volume in the form of a cavity. In some embodiments, the system further comprises a compliant surface (e.g., compliant membrane) at least partially enclosing (partially or completely enclosing) the interior volume of the chamber. For example, the chamber may comprise a bottom portion, side portions, and a compliant surface that together define an interior volume in the form of a cavity (e.g., that may be at least partially occupied by electrodes and/or electrolyte).

The chamber may be fabricated using any of a variety of techniques and using any of a variety of materials. For example, the chamber may be made of polymeric materials (e.g., plastics), composite materials, and combinations thereof. The chamber may be constructed using techniques known to one of ordinary skill in the art, including molding, milling, machining, additive manufacturing (e.g., 3D printing), and combinations thereof. In some, but not necessarily all embodiments, the first electrode and/or the second electrode are exposed to the interior volume (e.g., a same interior volume) of the chamber.

The chamber may have a relatively small volume. Having a small volume may be useful in some applications, such as those in which the electrochemical system is configured to be worn by a patient (e.g., as part of a device for drug infusion). In some embodiments, the chamber has a volume of less than or equal to 50 mL, less than or equal to 20 mL, less than or equal to 10 mL, less than or equal to 5 mL, less than or equal to 3 mL, less than or equal to 2 mL, less than or equal to 1 mL, less than or equal to 0.75 mL, less than or equal to 0.5 mL, less than or equal to 0.25 mL, less than or equal to 0.1 mL, or less. In some embodiments, the chamber has a volume of greater than or equal to 0.05 mL, greater than or equal to 0.1 mL, greater than or equal to 0.2 mL, greater than or equal to 0.3 mL, greater than or equal to 0.5 mL, greater than or equal to 1 mL, greater than or equal to 1.5 mL, greater than or equal to 2 mL, greater than or equal to 2.5 mL, greater than or equal to 3 mL, or greater. Combinations of these ranges (e.g., greater than or equal to 0.05 mL and less than or equal to 50 mL, greater than or equal to 0.05 mL and less than or equal to 5 mL) are possible.

As mentioned above, in some embodiments, a component of the electrochemical system (e.g., a chamber) comprises a compliant surface. In some embodiments in which a compliant surface is present, the compliant surface is in the form of a compliant membrane. The compliant surface may be in the form of a layer of material. The compliant surface (e.g., compliant membrane) may be configured or chosen to be deformable upon experiencing a sufficient magnitude of pressure (e.g., from generated gaseous species in the chamber). Any of a number of materials may be employed for the compliant surface. In some embodiments, the compliant surface comprises a soft material. In some embodiments, the compliant surface comprises a polymeric material. In some embodiments, the compliant surface (e.g., compliant membrane) has a low (or no) oxygen gas permeability (e.g., on a timescale of days or weeks or longer). In some embodiments, a chamber may be fully sealed with a compliant surface (e.g., with respect to fluids such as liquids).

In some embodiments, the compliant surface (e.g., compliant membrane) is configured to deform upon experiencing sufficient pressure. In some embodiments, a pressure sufficient to deform the compliant surface is greater than or equal to 5 kPa, greater than or equal to 10 kPa, greater than or equal to 15 kPa, greater than or equal to 20 kPa, greater than or equal to 25 kPa, greater than or equal to 30 kPa, greater than or equal to 35 kPa, greater than or equal to 40 kPa, greater than or equal 50 kPa, or greater. In some embodiments, a pressure sufficient to deform the compliant surface is less than or equal to 100 kPa, less than or equal to 90 kPa, less than or equal to 80 kPa, less than or equal to 70 kPa, less than or equal to 60 kPa, less than or equal to 50 kPa, less than or equal to 40 kPa, less than or equal to 35 kPa, less than or equal to 30 kPa, less than or equal to 25 kPa, less than or equal to 20 kPa, less than or equal to 15 kPa, less than or equal to 10 kPa, or less. Combinations of these ranges (e.g., greater than or equal to 5 kPa and less than or equal to 100 kPa, greater than or equal to 10 kPa and less than or equal to 30 kPa, or greater than or equal to 30 kPa and less than or equal to 100 kPa) are possible.

In some embodiments, a relatively low external voltage may be applied across the first electrode and the second electrode. Application of a relatively low voltage may result in a more energy-efficient system, which may be useful, for example, in relatively small systems such as relatively small pumps and/or valves. Additionally or alternatively, application of a relatively low voltage may allow for undesirable reactivity to be mitigated or eliminated, while desirable reactivity may occur under the relatively low applied voltages. For example, under a given set of conditions (e.g., electrode materials, electrolyte composition, pH), applying relatively high voltages (e.g., greater than or equal to 3.5 V, greater than or equal to 4 V, or higher) may promote undesirable hydrogen gas formation (e.g., at a first electrode), whereas application of lower voltages may promote desirable ion intercalation and/or redox activity with little to no undesirable hydrogen formation. Such an approach stands in contrast to certain existing electrolysis approaches, where relatively high voltages are applied to drive as much gas generation (e.g., oxygen and/or hydrogen gas generation) as possible. An ability to apply relatively low voltages while still operating an electrochemical system (e.g., for gas generation/consumption and/or compliant surface deformation) may be promoted via judicious selection of electrode materials. For example, selecting electroactive materials for the first electrode and second electrode that result in relatively low equilibrium potential differences (e.g., open-circuit potentials), and/or materials resulting in relatively low overpotentials for desired gas generation or consumption reactions may allow for operation with relatively low applied voltages.

In some embodiments, methods described herein involve applying a magnitude of voltage across the first electrode and second electrode of less than or equal to 3 V, less than or equal to 2.8 V, less than or equal to 2.6 V, less than or equal to 2.4 V, less than or equal to 2.4 V, less than or equal to 2.2 V, less than or equal to 2.0 V, less than or equal to 1.8 V, less than or equal to 1.6 V, less than or equal to 1.4 V, less than or equal to 1.3 V, less than or equal to 1.2 V, less than or equal to 1.1 V, less than or equal to 1.0 V, less than or equal to 0.9 V, less than or equal to 0.8 V or less. In some embodiments, methods described herein involve applying a magnitude of voltage across the first electrode and second electrode of greater than or equal to 0 V, greater than or equal to 0.3 V, greater than or equal to 0.6 V, greater than or equal to 0.8 V, greater than or equal to 1.0 V, greater than or equal to 1.2 V, greater than or equal to 1.3 V, greater than or equal to 1.4 V, greater than or equal to 1.6 V, greater than or equal to 1.8 V, greater than or equal to 2.0 V, greater than or equal to 2.2 V, greater than or equal to 2.4 V, or higher. Combinations of these ranges (e.g., greater than or equal to 1.2 V and less than or equal to 3 V, greater than or equal to 1.4 V and less than or equal to 2.0 V, greater than or equal to 0 V and less than or equal to 1.6 V, greater than or equal to 0V and less than or equal to 1.3 V) are possible.

An amount of gas generated may depend on desired applications (e.g., a desired amount of deformation of a compliant membrane, a desired amount of fluid to be displaced when operating an actuator, etc.). In some embodiments, a volume of gaseous species generated during a gas generation reaction is greater than or equal to 5 µL, greater than or equal to 10 µL, greater than or equal to 15 µL, greater than or equal to 25 µL, greater than or equal to 35 µL, greater than or equal to 50 µL, greater than or equal to 75 µL, greater than or equal to 100 µL, greater than or equal to 150 µL, greater than or equal to 200 µL, greater than or equal to 350 µL, greater than or equal to 500 µL, greater than or equal to 1 mL, greater than or equal to 2 mL, greater than or equal to 3 mL, or greater. In some embodiments, a volume of gaseous species generated during a gas generation reaction is less than or equal to 5 mL, less than or equal to 4 mL, less than or equal to 3 mL, less than or equal to 2 mL, less than or equal to 1 mL, less than or equal to 750 µL, less than or equal to 500 µL, less than or equal to 400 µL, less than or equal to 300 µL, less than or equal to 200 µL, less than or equal to 100 µL, or less. Combinations of these ranges (e.g., greater than or equal to 5 µL and less than or equal to 5 mL) are possible.

In some embodiments, a relatively high percentage of any gaseous species generated in the electrochemical system (e.g., via electrochemical reaction) is a desired gas. For example, a relatively high percentage of all generated gaseous species may be oxygen gas. This may be advantageous in some embodiments, where it is desired that oxygen gas be formed and relatively little to no hydrogen gas is generated (e.g., because hydrogen gas may be able to diffuse out of the system more readily than oxygen gas). In some embodiments, greater than or equal to 80 mol%, greater than or equal to 85 mol%, greater than or equal to 90 mol%, greater than or equal to 95 mol%, greater than or equal to 98 mol%, greater than or equal to 99 mol%, greater than or equal to 99.9 mol%, or more of a total amount of gas generated in the system (e.g., the chamber) during application of a voltage is oxygen gas. A relatively high percentage of generated gas being oxygen gas may be accomplished, for example, by selecting first electrodes to undergo non-gas-generating reactions such as ion intercalation reactions or redox reactions that simply change oxidation states of ions (e.g., manganese or iron ions).

In some embodiments in which a gaseous species is generated electrochemically, the system may be configured to limit or avoid a spontaneous/thermal “back reaction” in which the electrochemical reaction products, including the gaseous species, react and consume the generated gaseous species in a manner not driven by an applied voltage (e.g., when no electrical current is passed between the first electrode and second electrode). Avoiding such a back reaction (e.g., reaction of hydrogen gas and oxygen gas) may be desirable in some embodiments in which gas generation and consumption is electrically controlled (which can be useful in some applications such as drug delivery in which it is desirable for actuator shape/volume changes to be precisely controlled and metered via electrical measurements). A back reaction of electrochemical reaction products may be avoided, for example, by avoiding the use of materials that may catalyze the back reaction, by using kinetic control (where the system is configured such that the back reaction is kinetically slow), by avoiding generating non-desired gas (such as hydrogen), or by separating electrochemical reaction products such that they do not react. It has been realized that when a back reaction involves a surface reaction (e.g., a heterogeneous reaction), there may be a relatively high kinetic barrier for the back reaction, thereby slowing the back reaction. The reaction products may be separated, for example, using a separator between the first electrode and the second electrode. For example, a separator comprising a solid membrane that conducts light-mass ions (H⁺, OH⁻, Li⁺, Na⁺, K⁺, Mg²⁺, Ca²⁺) but not O₂(aq) can be added to the system (e.g., like the separator drawn in FIG. 5). For example, Nafion is H⁺ conducting, but does not allow O₂(aq) to cross over. Also, Li⁺/Na⁺ conducting solid electrolytes like NASICON, LIPON, PEO, LLZO, LGPS, etc. can be used. These solid membranes may be arranged to seal well against the chamber (e.g., against the chamber containing electrode A in FIG. 5, to prevent O₂ from reaching the first electrode (e.g., getting to electrode B in FIG. 5).

In some embodiments, the electrochemical system is configured such that when no electrical current is passed between the first electrode and the second electrode, a gas consumption reaction does not occur at the first electrode or the gas consumption reaction occurs at a rate of less than or equal to 5%, less than or equal to 3%, less than or equal to 2%, less than or equal to 1%, less than or equal to 0.5%, less than or equal to 0.3%, less than or equal to 0.2%, less than or equal to 0.1 mol%, less than or equal to 0.05 mol%, less than or equal to 0.02 mol%, less than or equal to 0.01 mol%, less than or equal to 0.005 mol%, less than or equal to 0.002 mol%, less than or equal to 0.0005 mol%, less than or equal to 0.0002 mol%, less than or equal to 0.0001 mol% per day, or less.

In some embodiments, consumption of generated gaseous species may be determined. Such a quantitative determination may allow for precise monitoring of gas consumption, which be important for precise determination of changes in volume of the system (e.g., when the presence of gaseous species can cause volume changes such as by deforming compliant surfaces). By monitoring gas consumption and/or volume changes, a reversible, controllable electrochemical actuator may be provided. In some embodiments, a method comprises passing a first current through the first electrode and second electrode for a first period of time such that a gaseous species is generated; deforming the compliant surface using the generated gaseous species; passing a second current through the first electrode and second electrode for a second period of time such that a portion of the gaseous species is consumed; and determining an amount of the gaseous species consumed during the passing of the second current. Determination of an amount of gaseous species consumed during the passage of the current may be performed using at least one electrochemical measurement. Determination of an amount of gaseous species consumed during the passage of the current may, for example, be accomplished by knowing a Faradaic efficiency for the gas consumption reaction (e.g., by performing a calibration experiment) and then measuring an amount of current passed through the first electrode during the gas consumption reaction. The first current and second current may be controlled, for example, by changing applied voltages (e.g., to reverse polarities of the first and second electrodes).

U.S. Provisional Pat. Application Serial No. 63/086,647, filed Oct. 2, 2020, and entitled “Electrochemical Systems and Methods,” is incorporated herein by reference in its entirety for all purposes.

The following examples are intended to illustrate certain embodiments of the present invention, but do not exemplify the full scope of the invention.

### EXAMPLE 1

### Introduction

This Example describes implementations of metal and metal-oxide electrodes immersed in an aqueous medium to reversibly release and consume O₂ molecules, to induce reversible volume changes of an encapsulated soft electrochemical cell with relatively low driving voltage (~1 V) and relatively low electrical energy expenditures (>100 mL/Wh). The goal of this embodiment was to precisely control the evolution and consumption of O₂ gas in a system that changes the total volume of the liquid electrolyte/solid electrodes/gas mixture. An actuator was formed by encapsulating the electrolyte/electrodes/gas mixture with a compliant membrane. While there are many membrane materials with low O₂ permeability, it is very difficult to keep generated H₂ encapsulated. Thus, in this embodiment, no H₂ gas is generated by design. The actuator can be used, for example, for pumping or valving purposes. This Example demonstrates the development of an electrically powered actuator with long storage stability, low driving voltage V=U^{A}-U^{B} where U^{A}, U^{B} are the electrical potentials on the two working electrodes A and B, high control authority, reversibility (e.g., the encapsulated system can expand and shrink multiple times, and if and only if when an external electrical current is given), low cost (e.g., using only earth-abundant elements), and bio-friendliness.

### Electrochemistry of the Oxygen-Only Actuation System

For reasons explained above, while water electrolysis reaction 2H₂O(aq) → 2H₂(gas) + O₂(gas) is generally well-known and has in some instances been used for the purpose of microfluidic pumping, the embodiment in this Example focuses on pure O₂ evolution/removal without generation of any hydrogen gas. It also focuses on reversible production and consumption of pure O₂ for two-way actuation, in contrast to certain existing technologies that focus only on controlled gas production / expansion, but not electrically controlled consumption / shrinkage, and not with reversibility / cyclability. Also different in this system from traditional water electrolysis systems is that only one (A) of the two solid electrodes A-B releases/consumes gaseous species:

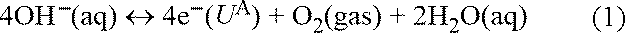

and the other electrode, B, while consuming electrons (e⁻) sent through the outer circuit from A, compensate by the dissolution or incorporation of ions such as light-mass ions (H⁺, OH⁻, Li⁺, Na⁺, K⁺, Mg²⁺, Ca²⁺) to/from the aqueous electrolyte and simultaneous change of valence of some transition-metal (TM) elements such as Mn and Fe of the solid oxide. In particular, this embodiment focuses on the redox reaction at electrode B (corresponding to the “first electrode” described above) provided by transition-metal elements Mn and Fe, as these are the biofriendly transition-metal elements (compared to Co, Ni, V, Cr, etc. which can be toxic), with equilibrium electrode potential of the B-side reactions close to that of the oxygen evolution reaction/oxygen reduction reaction (OER/ORR) at A-side, thus requiring much less driving voltage |V=U^{A}-U^{B}| than that required to drive standard water electrolysis (in theory the equilibrium cell potential is V^{eq}=1.23 V, but in practice V~2 V to 3 V due to kinetic overpotential). Another potentially important aspect in this embodiment is the design of the aqueous electrolyte, since the electrolyte pH and salt concentrations can also control the thermodynamic open-circuit voltage for U^{A,eq}, U^{B,eq}, as well as the kinetic polarization losses. In some applications, the electrolyte/electrodes system should possess long stability in storage at different temperatures, and low kinetic loss. In some instances, significant electrochemical cycle life can also be achieved in order to achieve repeated reversible expansion/shrinkage.

The setup of the encapsulated electrochemical soft cell is shown in FIG. 5, which shows a cross-sectional schematic illustration. Electrode A (corresponding to the “second electrode” described above) is responsible for oxygen evolution and reduction reactions (OER and ORR, respectively). Electrode B is responsible for substantially gas-free redox reactions using a transition-metal oxide composite electrode, which uses the redox reactions of lithium/sodium/proton ion intercalation materials to compensate for the electronic flow in outer circuit from A. The electrochemical cell was fully sealed with a soft deformable membrane. The membrane was chosen to be impermeable to O₂ over the timescale of days to weeks. For the usage as a pump, the soft but expandable electrochemical cell can be connected to another soft chamber that has a certain volume of fluid that needs to be pumped. When the volume of the soft electrochemical cell changes, the volume of the connected chamber will change correspondingly. For valving applications, the valve can open and close according to the volume change of the soft electrochemical cell.

The electrolyte chosen was water-based and at near-neutral pH or alkaline pH conditions, with salts such as LiOH, Li₂SO₄, Li₂ClO₄, LiTFSI, LiFSI, in the concentration range 0.1 M - 10 M. Alkaline electrolyte was observed to work better because of the lower equilibrium potential (U^{A,eq}) and overpotential (η^{A}≡|U^{A}-U^{A,eq}|)for OER and ORR. More importantly, transition metal redox reactions were realized to be more stable in an alkaline electrolyte with less transition-metal ion dissolution into the liquid solution than in other pH regimes.

The equilibrium potential of the transition-metal oxide was chosen to be more positive than that of the hydrogen evolution reaction at B, so that during the oxygen generation process at electrode A and with the flow of electrons to electrode B, the transition metal reduction will happen before HER occurs. Thus, in this embodiment the oxygen gas is the only gaseous species generated during the full-cell charging process, and the volume of the cell increases due to O₂ generation around electrode A. During the full cell discharge process, O₂ reduction will happen on electrode A also, leading to cell-volume decrease.

### Electrode A Material Selection

The electrode A materials are OER and ORR catalysts coated on a porous metal current collector that provided structural support and electronic percolation. O₂ molecules generated at A’s interface with electrolyte can exist in multiple forms in accordance with this embodiment. O₂(aq) means oxygen molecules dissolved in the liquid electrolyte; O₂(attached bubble) means O₂ residing in a gas bubble which is still attached to electrode A; O₂(detached bubble) means O₂ in a gas bubble no longer attached to electrode A. Generating O₂(aq), O₂(attached bubble) and O₂(detached bubble) can all increase the total volume of the subsystem (and thereby contribute to actuation). However, it is believed that O₂(attached bubble) is, in some cases, a kinetically beneficial form for reversible actuation because an O₂ gas bubble still attached to electrode A can undergo catalyzed ORR right away when the current is reversed, whereas O₂(aq) needs to diffuse back to contact electrode A to be reduced, and O₂(detached bubble) cannot be reduced except via O₂(aq) diffusion in the liquid electrolyte (shuttling), which tends to be slow compared to timescales desired in some embodiments. Thus, in this embodiment, electrode A was designed to have porous structures, which can confine the electrochemically generated oxygen as O₂(attached bubble) by forces such as capillary forces (liquid-gas, A-gas, and liquid-A interfaces) and the porous geometry.

It has been realized that in some instances, if the system is not carefully designed, O₂(aq) and O₂(detached bubble) may also contact electrode B, which can cause unwanted side reactions on electrode B. At O₂ partial pressure P_{O2}=1 atm, the solubility of O₂(aq) in 25° C. water is 40 mg(O₂)/L(water). This dissolved O₂(aq) can cause open-circuit oxidation on electrode B, which in principle can sag the total volume without external current control. However, this back reaction of electrode B with O₂(g) was observed to be practically quite slow, because the amount of dissolved O₂ in water is small compared to electrode B amount (1.25 nmol in a 1 mL chamber at 25° C., and the electrode B amount in such a chamber can be 0.5 mmol), and the reaction can only happen at the interface of electrode B and electrolyte. For some applications, such as medical devices that only actuate on timescales of several days to a week, this slow side reaction is usually not a problem. But if one wants to significantly reduce or even completely prevent such cross-over of O₂(aq) and shuttling-induced back reaction from happening, a solid membrane that conducts light-mass ions (H⁺, OH⁻, Li⁺, Na⁺, K⁺, Mg²⁺, Ca²⁺) but not O₂(aq) can be added to the separator drawn in FIG. 5. For example, Nafion is H⁺ conducting, but does not allow O₂(aq) to cross over from chamber A to chamber B, as this is the requirement for proton electrolyte fuel cells also. Also, Li⁺/Na⁺ conducting solid electrolyte like NASICON, LIPON, PEO, LLZO, LGPS, etc. can be used. These solid membranes need to seal well against the chamber containing electrode A in FIG. 5, to prevent O₂(aq) and O₂(detached bubble) from getting to electrode B.

Another way to inhibit the side reaction of electrode B with O₂ is to do surface coating on electrode B. Surface coating like Nafion, PEO, etc. can be used. These surface coatings are also solid electrolyte that works in essentially the same way as the solid-electrolyte separator above.

It was realized that for a given total current and rate of OER or ORR, the porous structure of electrode A can decrease the overpotential by increasing the electrochemical surface area, which lowers the electrical energy consumption per volume change. A porous stainless-steel foam (e.g., 304, 316, 316 L type stainless steel) or a titanium foam as the porous metal substrate is suitable for the harsh environment. The pore size can range from 50 nm to 500 µm with open porosity higher than 40%. For the high-current OER/ORR (i.e. a fast O₂ generation/consumption speed), a porous metal with a large surface area substrate can be beneficial.

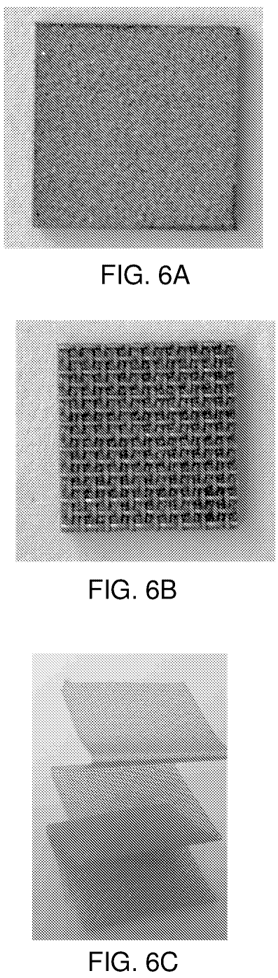

FIGS. 6A-6C shows images of porous electrodes employed in this Example. The porous electrode can be sintered powdered foam (FIG. 6A), sintered mesh foam (FIG. 6B) or folded mesh (FIG. 6C), made of, for example, 316L stainless steel. For low current (a slow oxygen generation speed), stainless-steel mesh and Ti mesh also work, in accordance with some embodiments. With different OER/ORR speed and oxygen amount, the requirement for pore size may quite different.

Noble metal catalysts were used on the porous substrates of electrode A in this embodiment. Noble metal catalysts can contribute to better OER and ORR catalyst performance at electrode A. Noble metals such as Platinum, Palladium, Iridium, Ruthenium, and their combinations, or their oxides (including Tantalum, Niobium oxides), could work in some embodiments. For example, Pt coated porous titanium, Pt coated porous stainless steel, mixed metal oxide coated titanium foam, Pt-mixed metal oxide coated titanium mesh, Iridium coated titanium foam, etc. can be suitable choices for electrode A. Doping by nitrogen, mercury, etc. is also useful as it can suppress hydrogen evolution reaction (HER), which is a parasitic side reaction that can happen at certain voltages. The coating method can be varied. Electrochemical plating, electroless plating, high-temperature sintering, sputtering, chemical vapor deposition, or atomic layer deposition are good choices. Different metallic substrates may require different coating or deposition methods of the catalyst.

Moreover, additional Nafion coating on noble metal-metal foam electrode A can improve the interface of oxygen and the electrode, which improves the ORR efficiently in some embodiments. A thin Nafion layer coating can be obtained by drop-coating using the commercial Nafion solution.

In a particular implementation, Pt was coated on 316L stainless steel foam by an electrochemical plating process. H₂PtCl₆. 6H₂O was used as the Pt precursor in an H₂SO₄ solution. Pt metallic catalysts were coated using cyclic voltammetry, or pulse current/voltage method.

Alternatively, for electroless deposition, a thin layer of copper or nickel or titanium/nickel was first coated on the stainless-steel foam. A replacement reaction with H₂PtCl₆ or some noble metal salt followed, to deposit metallic Pt. Atomic layer deposition (ALD), sputtering, and high-temperature sintering method can also be efficient for Pt loading. Especially, ALD was observed to control deposit 1-2 nm thick Pt, which can decrease the materials cost.

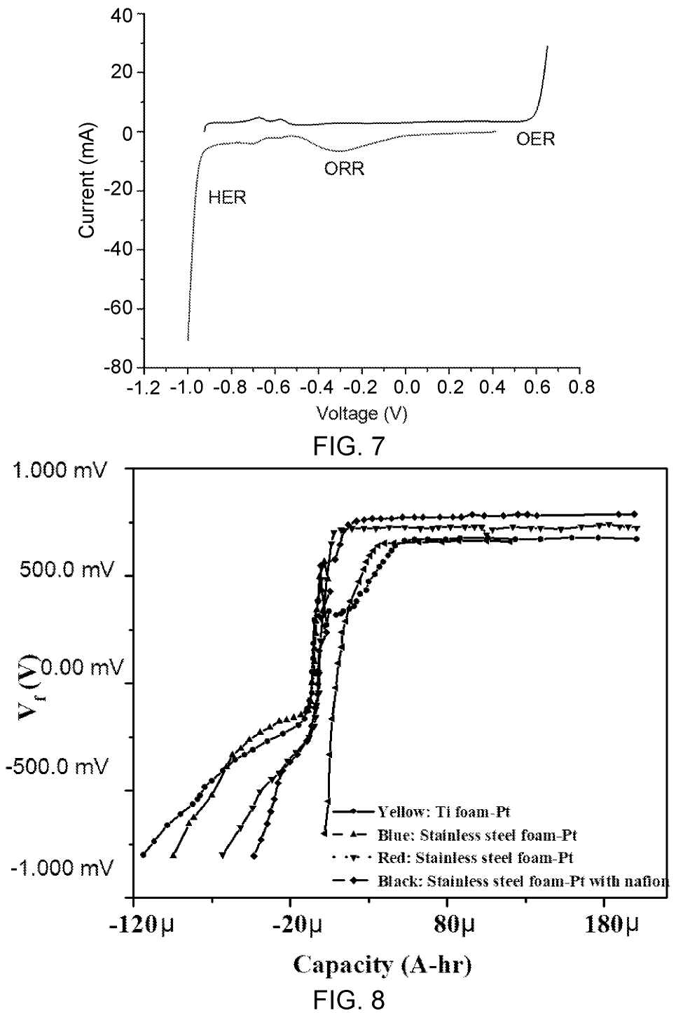

FIG. 7 shows the typical OER and ORR performance of a catalyst on porous electrode A. In particular, electrode A was a Pt-coated mixed metal oxide (MMO) substrate. The performance was tested in an aqueous 2 M LiOH electrolyte. The voltage in FIG. 7 is versus Hg/HgO. The typical potential for OER was +0.6 V versus Hg/HgO, and for ORR -0.2 V versus Hg/HgO. For high current OER and ORR, metal foam worked better. FIG. 8 shows a plot of OER and ORR performance of the catalyst charge and discharge at 10 mA and 2 mA. The electrode area was around 0.5-0.6 cm². As shown in FIG. 8, stainless steel foam coated with Pt and Nafion showed higher ORR efficiency than the others. Fast speed of generating and consuming can be beneficial in some embodiments, as it can cause fast volume change, which corresponding to the fast liquid delivery or fast speed valve open and close.

### Electrode B Materials Selection

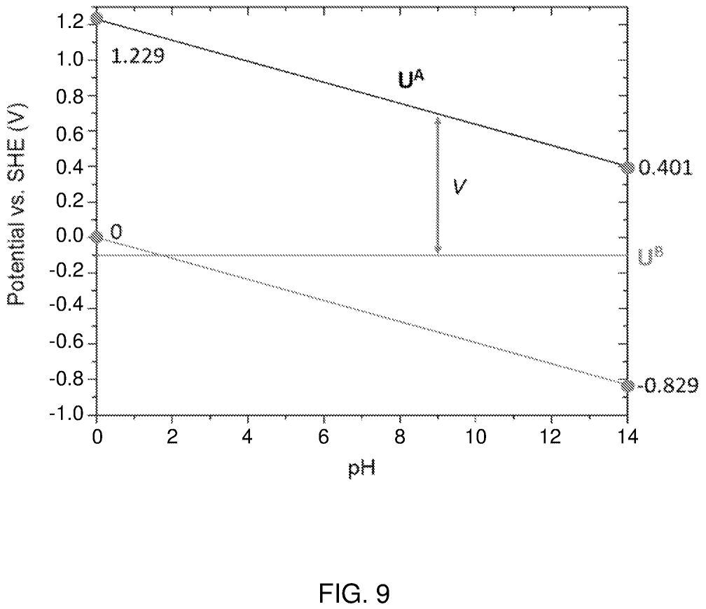

The intercalation material selection for electrode B in the embodiments of this Example were made, in some instances, based on thermodynamic and kinetic considerations relating to the electrode B chemistry and the OER/ORR reactions. FIG. 9 shows a Pourbaix diagram of water splitting and U^{B} of the intercalation electrode B. In FIG. 9, the horizontal line is a pH-independent solid reaction potential U^{B}.The upper curve is the pH-dependent OER/ORR U^{A,eq} and the lower curve is the pH-dependent HER potential. The smaller the V=U^{A}-U^{B} (difference between the upper curve and the lower curve at a specific pH (specific x-axis value)), the lower the voltage and energy needed when generating oxygen. In this embodiment, where HER is considered undesirable, the reduction potential for the electrode B material should be higher than that of HER under operative conditions, so that ORR occurs before HER and trouble-making H₂ generation is reduced or eliminated. Thus, higher pH (alkaline) aqueous electrolyte was employed to improve the main device figure-of-merit (FOM), defined as the volume change divided by electrical energy expenditure in this Example.

A series of redox reactions located between the potential of HER and OER are listed below. The standard electrode potential can provide guidance, in combination with the insight of the present disclosure, to choose some material systems, and for those reactions that are dependent on pH, one should also check the Pourbaix diagram of those material systems.

For the open-circuit U^{B}, exemplary redox reactions are listed below:

| | |
|---|---|
| MnO₂(s) + 4H⁺ + 2e⁻ ⇋ Mn²⁺ + 2H₂O | +1.23 V |
| Ag₂O(s) + 2H⁺+ 2e⁻ ⇋ 2Ag(s) + H₂O | +1.17 V |
| MnO2(s) + 4H⁺+ e- ⇋ Mn³+ + 2H₂O | +0.95 V |
| Fe³⁺ + e⁻ ⇋ Fe²⁺ | +0.77 V |
| 1,4-Benzoquinone + 2H⁺ +2e⁻⇋ Hydroquinone | +0.6992 V |
| Fc⁺+ e⁻ ⇋ Fc(s) | +0.641 V |
| I₂(s) + 2e- ⇋ 2I⁻ | +0.54 V |
| [Fe(CN)₆]³⁻ + e- ⇋ [Fe(CN)₆]⁴⁻ | +0.36 V |
| Cu²⁺ + 2e⁻ ⇋ Cu(s) | +0.340 V |
| VO²⁺ + 2H⁺ + e- ⇋ V³⁺ + H₂O | +0.34 V |
| Bi³⁺ +3e⁻ ⇋ Bi(s) | +0.32 V |
| Ge⁴⁺ + 4e⁻ ⇋ Ge(s) | +0.12 V |
| H₂MoO₄(aq) + 6H⁺ + 6e⁻ ⇋ Mo(s) + 4 H₂O | +0.11 V |
| Fe³⁺ + 3e⁻⁻ ⇋ Fe(s) | -0.04 V |
| WO₃(aq) + 6H⁺+ 6e⁻ ⇋ W(s) + 3H₂O | -0.09 V |
| SnO₂(s) + 2H+ + 2e- ⇋ SnO(s) + H₂O | -0.09 V |
| SnO(s) + 2H+ + 2e- ⇋ Sn(s) + H₂O | -0.10 V |
| WO₂(s) + 4H⁺ + 4e⁻ ⇋ W(s) + 2H₂O | -0.12 V |
| Pb²⁺ + 2e⁻ ⇋ Pb(s) | -0.13 V |
| Sn²⁺ + 2e⁻ ⇋ Sn(s) | -0.13 V |
| MoO₂(s) + 4H⁺ + 4e⁻ ⇋ Mo(s) + 2H₂O | -0.15 V |
| V³⁺ + e⁻ ⇋ V²⁺ | -0.26 V |
| Ni²⁺ + 2e⁻ ⇋ Ni(s) | -0.25 V |
| PbSO₄(s) + 2e⁻ ⇋ Pb(s) + SO42- | -0.3588 V |
| Fe²⁺ + 2e⁻ ⇋ Fe(s) | -0.44 V |

where the potential is with respect to the Standard Hydrogen Electrode (SHE). SHE is 3.05 V above the Li⁺/Li reference and -0.174 V below the Hg/HgO (0.1 M KOH) reference.

It is noted that the Table above provides only rough guidance on U^{B} design (which is aimed to be as close to U^{A} as possible in this Example, when both are defined with respect to the standard hydrogen electrode potential SHE). With the different atomic coordination environments of the redox-active transition metal (TM) in electrode B, the standard electrode potential can change significantly. The selection of the materials may also dependent on the desired application of the electrochemical system. For example, for use in a one-way gas-driven pump, no reversible electrochemical reaction may be required, in which case the reduction of MnO₂ to Mn²⁺, Cu²⁺ to Cu, SnO₂ to Sn, etc. can be chosen. The working pH may be chosen with guidance from the Pourbaix diagram in FIG. 9.

Below, this Example further focuses on examples of reversible reactions of the type

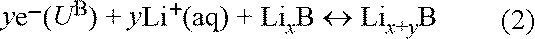

that uses solid-state lithium ion intercalation reactions. Chemical group B (of solid electrode B) in this embodiment will undergo valence change. It may be beneficial that such a valence change happen with limited to no change in the overall crystal structure and bonding topology of the material, (e.g., a solid-state intercalation reaction). Volume change of the lattice should also be relatively low in this embodiment, which can reduce mechanical stress in cycling and prolong the stability of the electrode B. The Li element in reaction (2) can be replaced by other ions such as Na⁺ H⁺. There are many possible candidates for B, but this embodiment employs reactions (2) having as small open-circuit |U^{A}-U^{B}| as possible in reference to reaction (1). Other considerations included using materials that are cheap, biofriendly, and processable.

A series of Li⁺, Na⁺ and proton-intercalation materials were used in this Example based on the redox reaction with multi-valent transition-metal cations Mn, Fe (and V, Co). For lithium-ion intercalation, MnO₂, LiMn₂O₄, LiₓMnO₂, Li₄Mn₅O₁₂, LiFePO₄, FePO₄, V₂O₅, Li₃V₂(PO4)₃, LiCoO₂, Prussian white analogues (KₓM_{y}[Fe(CN)₆]_{z} (M = Fe, Co, Ni, Cu)) and Prussian blue (KFe[Fe(CN)₆]), etc. could be used. The electrolyte may contain Li (or Na, proton) salt, such as LiOH, Li₂SO₄, Li₂ClO₄, LiTFSI, LiFSI, etc. The concentration of the lithium salt can be varied, which is depending on the material we use, but it generally ranges from 0.1 M to 10 M.

Upon increase of U^{A} and decrease of U^{B} (V↑), both reaction (1) and (2) proceed to the right-hand-side (RHS). As a result, the reaction will result in chamber A becoming more acidic due to (1), thus shifting pH towards the left on FIG. 8, which makes ORR slower. In Chamber B, the concentration of Li⁺(aq) will drop in the electrolyte, compensated by more H⁺(aq) in the electrolyte that crosses over from chamber B. The pH shift may depend on the volume and original pH of the electrolyte. The pressure change of the chamber is related to the volume of oxygen and the residual volume of air in the chamber. For example, for a 20 µL oxygen gas system which working as a valve, the consumption of OH⁻ may be around 3.5 µmol. The original total amount of OH⁻ is 4 mol with a total liquid electrolyte volume of 250 µL, then the OH⁻ concentration after OER is 3.98 mol. Thus, the pH remains nearly the same after OER for practical cells.

It has been realized that for reaction (2) to readily happen toward the RHS in the very first volume expansion (i.e., first use of the device), there must be vacant atomic sites for Li in LiₓB to make Li_{x+y}B. Electrode materials like MnO₂, LiMn₂O₄, Li₄Mn₅O₁₂, FePO₄, V₂O₅, etc. have vacant atomic sites and thus can be used in such electrochemical reduction-first mode.

Alternatively, one can achieve LiₓB not directly in chemical synthesis, but by electrochemically delithiating materials like LiFePO₄ that comes with a full complement of lithium with no vacant sites, to make

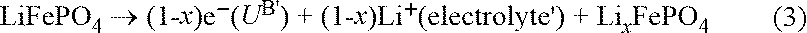

Note that reaction (3) may occur as a preparatory step a separate electrochemical system, in a liquid electrolyte that may differ from the final aqueous electrolyte used in chamber B in service (e.g., pumping, valving). (3) is a pre-processing step, after which the different liquid electrolyte is washed away from the electrode. For example, materials like LiMn₂O₄, Li₄Mn₅O₁₂, LiFePO₄ can be slurry coated on the current collector and charged (delithiated) first by (3), washed, and then an entire electrode loaded with LiₓMn₂O₄, LiₓMn₅O₁₂, LiₓFePO₄ may be harvested, (e.g., divided by cutting, and assembled into the final device.

It should be noted that chemically synthesized compounds LiMn₂O₄, Li₄Mn₅O₁₂ can be used in both the charge-first mode as pre-processing (3), or the discharge-first mode (2) directly. This makes these materials versatile, since the open-circuit potential U^{B} can be tuned in a wide range of voltages and used to match U^{A} to reduce the open-circuit |U^{A}-U^{B}l. With LiMn₂O₄ and Li₄Mn₅O₁₂ one can drive gas generation with V=U^{A}-U^{B} in the range of V ~ 1 Volt or less, which is significantly lower than that used in certain existing electrochemical pumps. This means instead of using 2 or more commercial 2032-type button batteries to drive the pump, this embodiment can use just 1 2032-type button battery. The energy efficiency of creating/removing O₂ can be greatly improved by intentionally designing U^{B} to reduce |U^{A}-U^{B}|, evaluated in terms of the mole(O₂)/Wh metric.

Some corresponding sodium-bearing composites for sodium-ion intercalation may also be suitable for electrode B, to balance the electrochemically driven O₂ creation/removal at electrode A.

Electrode B can be manufactured by a slurry casting process. Typically, active materials (MnO₂, LiMn₂O₄, Li₄Mn₅O₁₂, FePO₄, V₂O₅, LiₓMn₂O₄, LiₓMn₅O₁₂, LiₓFePO₄, etc.), conductive agents such as carbon black or carbon nanotubes, and binders such as polyvinylidene difluoride (PVDF), polytetrafluoroethylene (PTFE), and/or polyimide (PI) were mixed to form a uniform slurry first. Then the slurry was cast on stainless steel mesh current collector. The current collector should be stable in the electrolyte and during the oxidation and reduction process. The use of metallic mesh here is to prevent spallation of the slurry from the current collector. The slurry can be detached easily in the electrolyte when using stainless steel foil as the current collector.

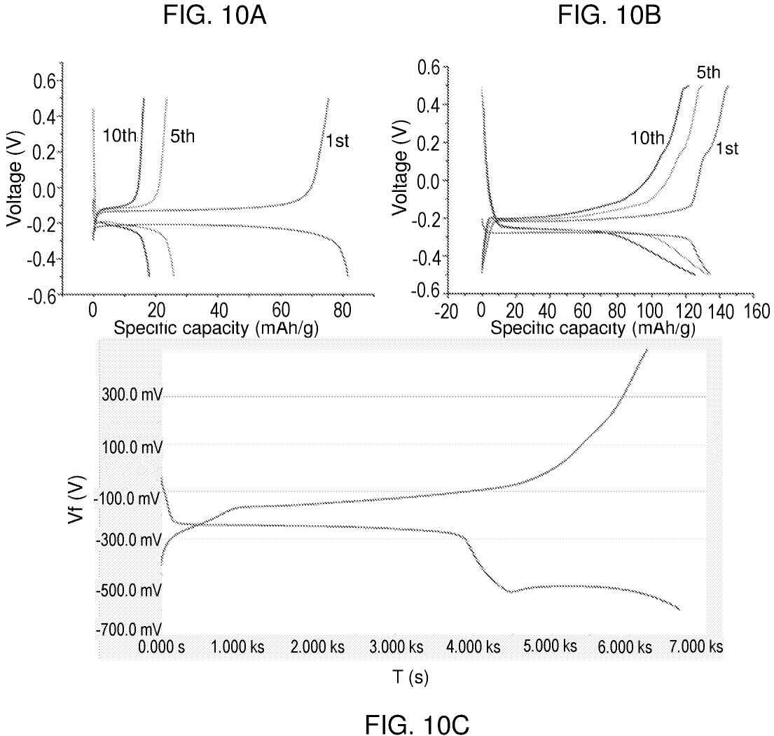

In a particular implementation, the charge and discharge profile of the LiMn₂O₄ and Li₄Mn₅O₁₂ materials in a 2 M LiOH electrolyte is shown in FIGS. 10A-10B, which was discharged first in a three-electrode system, with Pt as the counter electrode and Hg/HgO as a reference electrode. FIGS. 10A-10C shows charge and discharge curves of electrode B (FIG. 10A) LiMn₂O₄ and (FIG. 10B) Li₄Mn₅O₁₂ discharged to -0.5 V, and (FIG. 10C) Li₄Mn₅O₁₂ discharged to -0.6 V versus Hg/HgO reference electrode, at current density 100 mA/g(B), and loading 10 mg/cm². Here, these electrodes were used in the discharge-first mode, right after synthesis, without going through preprocessing like (3).

Both LiMn₂O₄ and Li₄Mn₅O₁₂ materials showed discharge plateau U^{B} around -0.25 to -0.2 V versus Hg/HgO, which is much larger than the HER voltage (around -0.9 V versus Hg/HgO). Therefore, reduction of LMO will occur before HER. This produces significant energy savings, and also removes the problem of the transiency of H₂ from the system. The charge plateau U^{B} was around -0.2 to -0.15 V. Thus, the polarization loss of both LiMn₂O₄ and Li₄Mn₅O₁₂ as working electrode B was small, implying the energy efficiency of the electrode was excellent. The porous Pt counter electrode A accepted the same total current as through the outer circuit with typical potential +0.6 V versus Hg/HgO for OER and -0.2 V versus Hg/HgO for ORR as shown in FIG. 7. Thus, the typical gas-driving voltage of the device was ~+0.6 V-(-0.2 V)=0.8 V to +0.6 V-(-0.25 V)=0.85 V for O₂ generation, if one uses the first plateau in FIG. 10A, FIG. 10B.

The cycling performance of Li₄Mn₅O₁₂ was observed to be much better than that of LiMn₂O₄ under the employed conditions. Li₄Mn₅O₁₂ also showed a much higher capacity than LiMn₂O₄. When discharged to -0.6 V vs. Hg/HgO, a new plateau showed up at around -0.5 V (see FIG. XC), which was still much higher than HER potential. So this second plateau at around -0.5 V versus Hg/HgO can also be used for electrode B if desirable. The Li₄Mn₅O₁₂ electrode can contribute 45% more capacity (~190 mAh/g) than when discharged to -0.5 V (the full-cell operating voltage would be ~+0.6 V-(-0.5 V)=1.1 V if one uses this second plateau of B).

The reaction during discharge and charge are listed below:

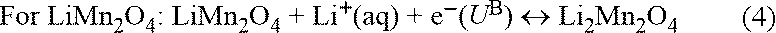

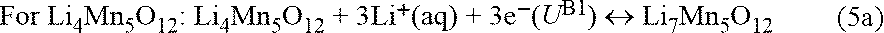

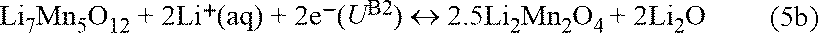

The first plateau of Li₄Mn₅O₁₂ at -0.25 V vs. Hg/HgO in FIG. 10C is believed to come from reaction (5a), and the second plateau at -0.5 V vs. Hg/HgO is believed to come from reaction (5b). To utilize the (5b) capacity, the device needs a bit higher driving voltage, i.e. +0.6 V(OER) - (-0.5 V(5b)) = 1.1 V. This still is well within the range of all single button-cell batteries.

For long-term cycling, some Mn dissolution into the electrolyte was observed, which is the main reason attributed to the cyclability fading of the device. However, increasing the pH, by for example enhancing LiOH concentration, can inhibit Mn cation dissolution into the liquid electrolyte and get better electrochemical performance. With an adjusted electrolyte of 4 M or saturated LiOH and 1 M Li₂SO₄, the discharge capacity of B can reach 210 mAh/g(B) with much better cycling stability.

### Separator Material Selection

A modified porous polypropylene (PP) separator (NKK-MPF30AC) which is stable in strong alkaline solution conditions is one choice of separator, to prevent electronic short-circuiting between electrode A and B. Full-cell performance was tested in an encapsulated system, leaving one hole open and connected to a transparent tube to observe the total volume change of this liquid electrolyte — solid electrodes — gas mixture during charge and discharge. As mentioned above, most polymeric materials are impermeable enough to O₂ to allow enough control authority over days to weeks.

As also explained above “Electrode A Material Selection”, to limit or prevent the cross-over of oxygen from Electrode A to Electrode B, a membrane that conducts light-mass ions (H⁺, OH⁻, Li⁺, Na⁺, K⁺, Mg²⁺, Ca²⁺) but not O₂(aq) can be used as an alternative to the more common porous separator (such as porous polypropylene separators used in lithium-ion batteries). For example, Nafion is H⁺ conducting, but does not conduct O₂(aq). Also, Li⁺/Na⁺ conducting solid electrolyte like NASICON, LIPON, PEO, LGPS, etc. can be used. These solid membranes need to seal well against the upper chamber in FIG. 5, to limit or prevent O₂(aq) and O₂(detached bubble) from getting to electrode B. In some embodiments involving actuation/continuous drug delivery applications longer than a week, this sealing is beneficial.

### Full Cell: Use Case

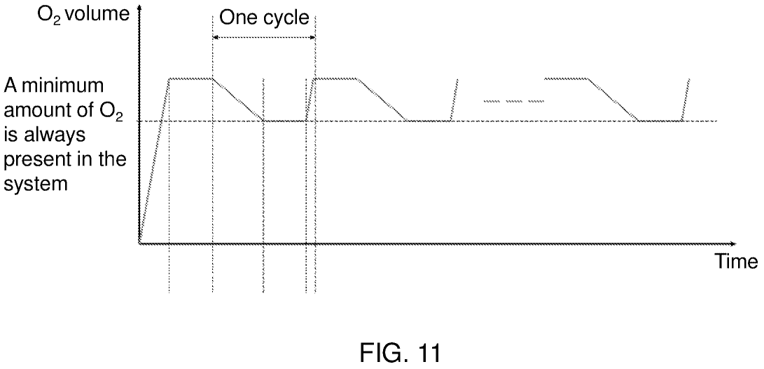

The electrochemistry presented above explains the reversibility of oxygen evolution and consumption. However, in actual use, completely consuming all oxygen generated may be kinetically slow. This is because it is kinetically slow to consume O₂(detached bubbles) — once the gas bubble has detached from electrode A, they need to be resorbed into water as O₂(aq) to be able to contact electrode A and undergo ORR. Therefore, in an actual use case, a design was employed such that not all evolved oxygen was depleted the reaction was reversed. FIG. 11 shows how a design of an actuator is cycled in principle. FIG. 11 shows a schematic of oxygen evolution and reduction volume over time according to this embodiment.

### Full Cell Performance Characterization: Volume Measurement

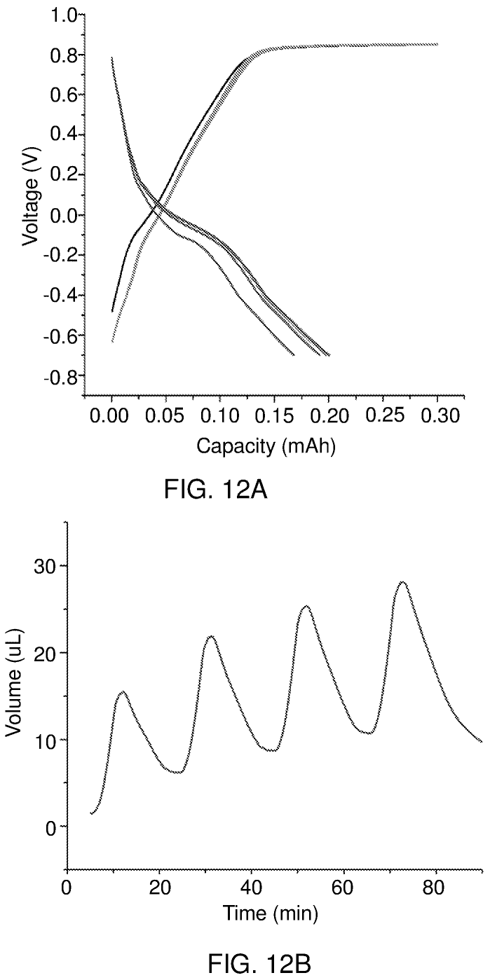

The charge (OER) and discharge (ORR) profile and the corresponding volume change of the full cell of this Example are shown in FIGS. 12A-12B. FIG. 12A shows charge and discharge profile of the full cell with a charge current of 2 mA and discharge current of 1 mA; FIG. 12B shows the volume change of the full cell. Electrode A was Pt-Stainless steel mesh, and electrode B was Li₄Mn₅O₁₂.It was observed that the full cell showed good cycling performance, and the corresponding total volume change was around 18 µL, for a total electrical charge of about 0.2 mAh. One can increase or decrease the volume by changing the charging /discharging capacity of electrode B.

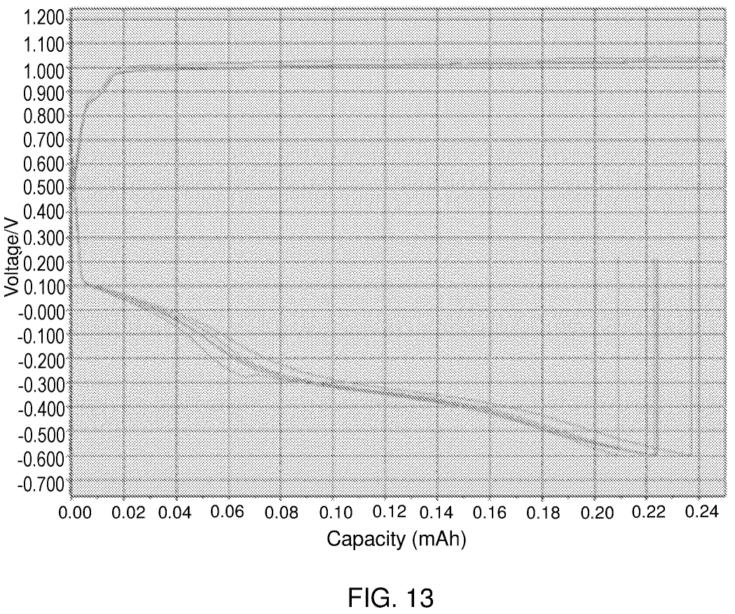

FIG. 13 shows the full-cell performance cycled at a charge current of 10 mA, and a discharge current of 1 mA. In particular, FIG. 13 shows cycling performance of Pt on stainless steel foam for electrode A, and Li₄Mn₅O₁₂ for electrode B, as a full cell in alkaline aqueous electrolyte. The charge plateau was around 1 V. The device was stably cycled with gas expansion and shrinkage for at least 10 cycles. This is a significant advance compared to certain existing technologies using gas molecules as actuation agents.

### Full Cell Performance Characterization: Pressure Measurement

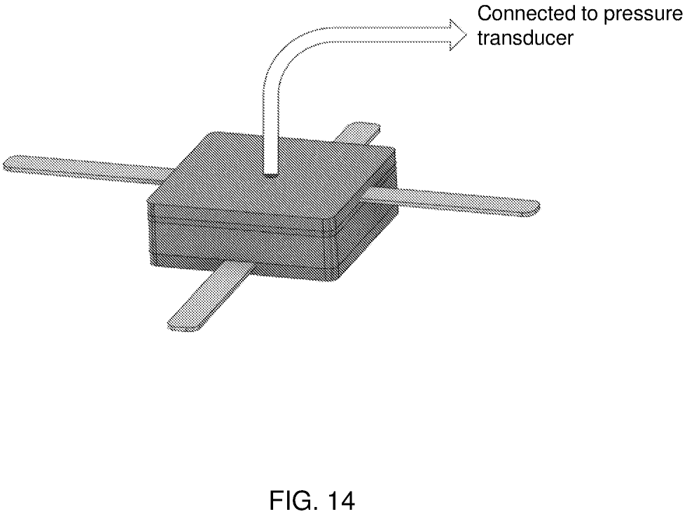

The electrochemical system described in this Example was connected to a pressure transducer (FIG. 14), to measure the pressure change during cycling of the full cell. FIG. 14 shows a perspective view schematic illustration of the electrochemical system connected to a pressure transducer.

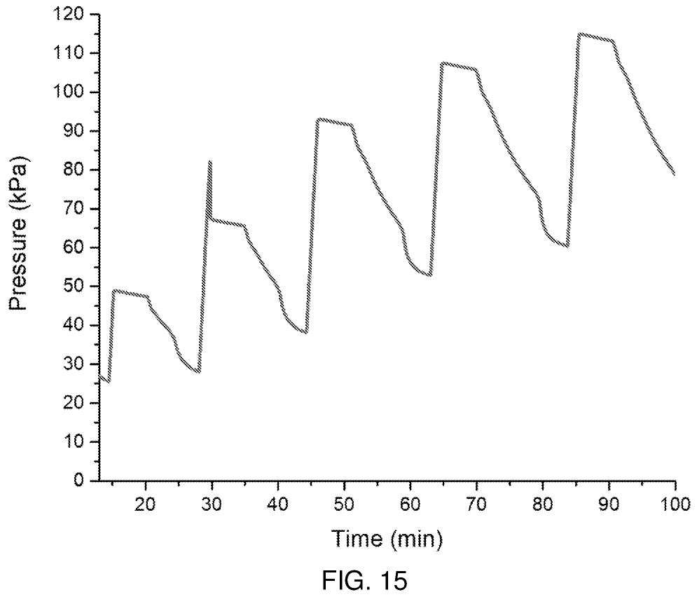

FIG. 15 shows the measured pressure change during charge and discharge of the full cell. The cell was rested for 5 min after each charge and discharge. The pressure was observed to go up and down for several cycles, indicating suitability for use as a valve. Also, the control system can be designed to adapt to the accumulation of pressure during cycling.

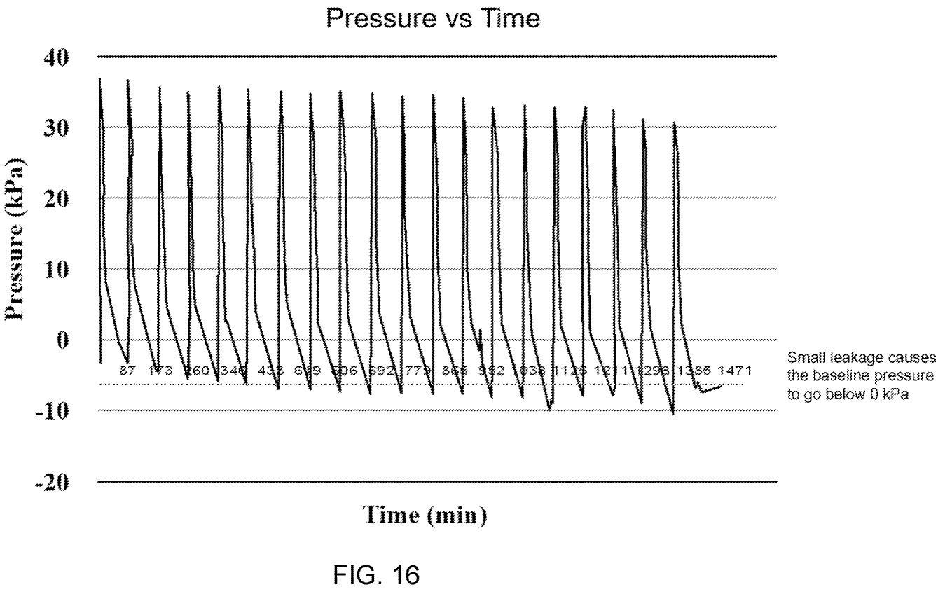

FIG. 16 shows pressure measurement for 20 cycles of the electrochemical system with the full cell using a Pt catalyst for electrode A formed by atomic layer deposition. ORR was observed to be much improved compared to that observed with electrochemically-deposited-Pt electrode A.

### Summary and Conclusions

According to equation (1), 4 electrons through the outer circuit creates one O₂ molecule, so 0.1 mAh charge capacity on electrode A creates 9.3278×10⁻⁷ mol(O₂), or 20.8944 µL STP (standard pressure and temperature) oxygen (at P_{O2}=1 atm and 300 K), as 1 mole of STP gas occupies 22.4 L. However, P_{O2} can significantly exceed 1 atm pressure inside the O₂(attached bubble) due to additional Laplace pressure AP=2γ/R_{bubble} due to capillarity, where the surface tension of liquid water γ =0.069 J/m². So if the bubble diameter R_{bubble}= 1 µm, ΔP is as large as 1.38 atm, and then the internal pressure P_{O2} is 2.38 atm, so the amount of volume expansion generated by same mole of O₂(attached bubble) is correspondingly smaller than the P_{O2}=1 atm estimate above. Adding to this, there are also some side reactions on electrode A, so the efficiency of oxygen generation will be lower than 20.8944 µL / 0.1 mAh. The side reactions are believed to mainly come from the corrosion and pseudocapacitance behavior from porous electrode A such as stainless steel.

In this Example, it was experimentally found that in order to generate 40 µL volume expansion/shrinkage by O₂, ~0.4 mAh electrical charge/discharge capacity was actually needed, so the actual figure-of-merit (FOM) of volume change divided by electrical energy expenditure is about 40 µL/0.4 mAh/0.8 V = 125 mL/Wh. The working voltage of the full device was generally substantially less than 1 V (see FIG. 11), albeit it generally also depended on the current density. To get the 40 µL oxygen, all that was needed was a battery that can deliver 0.4 mAh capacity and have a nameplate working voltage slightly higher than 1 V.

The volume expansion required for pump delivery of drugs such as insulin delivery is 2 mL at present, which ideally just requires a capacity of 10 mAh. Considering the side reactions on electrode A and P_{O2}(attached bubble)> 1 atm, a 20 –30 mAh capacity battery is already sufficient experimentally with the currently demonstrated experimental soft cell. Alkaline, lithium primary, and lithium secondary batteries are all suitable for such a system. The gas evolution rate was observed to depend on the current density. At 10 mA/cm²(nominal electrode A surface area), the oxygen generation rate was around 16 µL/min. The pressure increase rate is related to both the OER rate and the residual gas volume in the chamber. At present, the device of this Example can achieve 25 kPa/min at 10 mA/cm². By adjusting the electrolyte filling method, one can achieve a better vacuum in the cell chamber. Then, after filling the electrolyte, the residual gas volume before electrochemical actuation can be decreased further, and even better reversible actuation characteristics can be obtained.

In conclusion, this Example demonstrates the rational design and implementation of a class of pure-O₂ gas-driven actuators (pump and valve), that can reversibly expand and shrink in volume by generating/consuming O₂ molecules. Unlike H₂, there are many O₂-impermeable polymers that easily seal freshly generated O₂ in a soft cell for multiple days at least. The device has been designed such that per mL of volume expansion, it expends relatively little electrical energy, by matching the equilibrium potentials of the porous metal gas-electrode A current with solid-oxide electrode B (MnO₂, LiMn₂O₄, Li₄Mn₅O₁₂, FePO₄, V₂O₅, LiₓMn₂O₄, LiₓMn₅O₁₂, LiₓFePO₄, etc.), in aqueous electrolyte with neutral or alkaline pH, with salts such as LiOH, Li₂SO₄, Li₂ClO₄, LiTFSI, LiFSI, etc. These devices demonstrated what is believed to be first-of-a-kind (FOAK) reversibility in pressure and volume, for more than 10 cycles, and the actual figure-of-merit (FOM) of volume change divided by electrical energy used was around 40 µL/0.4 mAh/0.8 V = 125 mL/Wh. Thus, the electrochemical system of this Example can be driven by ~10¹ mAh capacity miniature batteries as long as the battery’s nameplate voltage is ~1 V. This class of electrochemically driven soft cells may be suitable in electronically controllable pumps and valves, e.g., for drug delivery and other applications.

### EXAMPLE 2

This Example describes the use of MnO₂ as a cathode to limit the production of hydrogen during the electrolysis of electrolyte solutions, which can lead to hydrogen-free pumping with beneficial storage and long-term operation stability.

It was observed that MnO₂ coated electrodes could be used for hydrogen-free electrolysis of aqueous electrolyte. Further, it was observed in this embodiment that lower pH values for the electrolyte better reduced the electrolysis voltage requirement. The combination of pH 3 phosphate solution and 15 mg/cm² MnO² coating successfully brought down the voltage requirement to be less than 1.2 V with very low hydrogen generation during the test. Compared to a Ag/AgCl system, the chemistry of this Example is more mass efficient (two electrons for MnO₂) and cost efficient (no noble metals).

In this experiment, the following material were used:
1. MnO₂ coated Ni backing electrodes with 4.2, 8.6 and 15 mg/cm² loading (double sided).
2. MnO₂ coated ss (25 µm) electrodes with 5, 12, 15 and 28 mg/cm² loading (single sided)
3. pH 3 1 M Phosphate solution and pH 8.5 1 M phosphate solution from Sigma
4. CH Instruments potentiostat
5. Laptop with Matlab
6. Electrolysis setup with mass balance
7. Customized cuvette, with needle and tubing.
8. SS sheet cut to fit the cuvette.

The electrode potentials at acidic conditions are listed in the table.

| | |
|---|---|
| Electrode reaction | Potential (V) |
| O₂(g)+4H⁺+4e⁻⇔2H₂O | 1.229 |
| MnO₂(s)+4H⁺+2e⁻⇔Mn²⁺+2H₂O | 1.23 V |

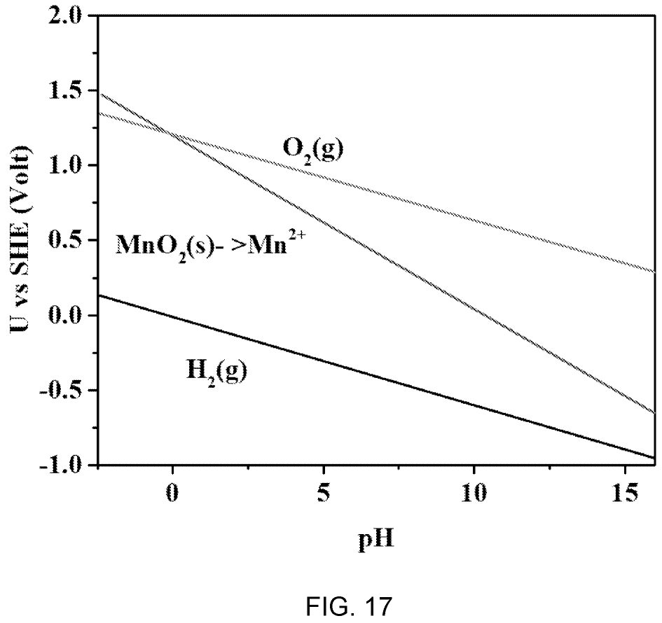

The changes of electrode potential with pH are shown in FIG. 17. In theory, under very acidic conditions (pH<0), MnO₂ could drive O₂ production spontaneously (without energetic input). However, due to the hazard concerns of low pH, the range of pH 3-4 determined to be a starting point. The theoretical electrolysis voltage is about 0.3 V and oxygen gas generation was observed for applied voltages of 0.6 V. As a comparison, pH 8.5 1 M phosphate buffer was also tested.

In some applications, the ionic strength is also a major factor affecting an actual voltage requirement. It was realized that higher ionic strength helps reduce non-Faradaic power consumption. Instead of using acetic acid, which is a mild acid with a strong smell, phosphate buffer was used. It was determined that there were several advantages with phosphate buffer: 1. Higher ionic strength; 2. No smell; 3. It bufferred well at pH 3-4 to keep the pH of the electrolyte buffer stable.

### Setup of Tests

1. Buffer preparation

| BUFFER: | To make 1000 ml of 1 M Phosphate (pKal=2.15) Buffer, pH=3, |
|---|---|
| | Ionic strength = 0.912 M, |
| | Thermodynamic pKₐ = 2.15, Apparent pKₐ‘ = 1.98 |
| | Temperature coefficient = 0.0044 per °C |
| | Prepared at 21° C., used at 21° C. |
| RECIPE: | • Dissolve 0.0877 mol of acid component |
| | • Dissolve 0.9122 mol of basic component |
| | • (No added neutral salts, ionic strength due to buffer alone.) |
| | • Make up to 1000 ml with pure water |
| Alternatively | • Dissolve 98 g of Phosphoric acid (Mr = 98) in approx. 900 ml of pure water. |
| | • (No added neutral salts, I due to buffer alone.) |
| | • Titrate to pH 3 at the lab temperature of 21° C. with monovalent strong base or acid as needed. |
| | • Make up volume to 1000 ml with pure water |
| | • Buffer will, of course, be pH 3 at 21° C. |

### 2. Chamber Preparation

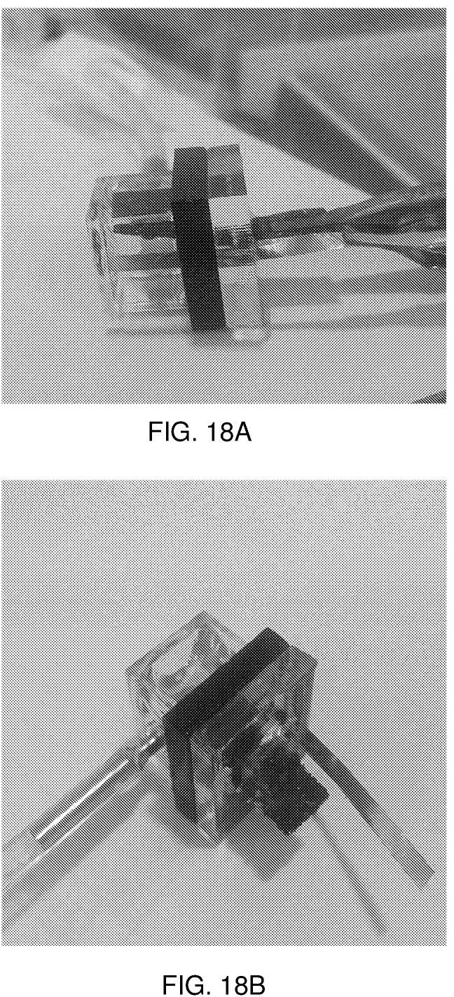

The electrolytic chamber was cut out of clear cuvette with about 1 cm height. The cuvette was assembled with acrylic caps with slots for electrode insertion. The MnO₂ electrodes were cut into 5 mm wide strips and stainless steel (ss) sheets were cut into 2 mm wide strips. An open port (1/16″) was drilled on the side and an ss needle was obtained from syringe needles. UV cure adhesive and acrylic glue were used to seal the whole chamber. Electrolyte was loaded with a long needle syringe. FIGS. 18A-18B show images of the assembled electrolytic chamber, with one port for electrolyte loading. Both electrodes were inserted through an open slot and sealed with UV cure adhesive.

Since the coating layer is porous, it has been noticed that even with secure sealing, electrolyte will still slowly wet through the layer and bypass the UV cure sealing. The balance reading will constantly drop and a salt layer around the electrode will be spotted. To mitigate this effect, the coating of the electrode out of the cuvette is to be scraped off and the UV cure adhesive is to cover both the electrode and the copper clip. Thus a more reliable measurement could be achieved.

### 3. Test Protocols

The filled cuvette was connected to a mass balance with ⅛ tubing. The tubing was primed. The CHI potentiostat was connected to the anode and cathode. Chronopotentiometry was used to control the electrolysis current. The electrolytic voltage was be monitored together with the mass balance reading (if recorded).

### Experiment and Results

### 1. Size Considerations for 250 µL Delivery

Assuming the coating is pure MnO₂ and 100% efficiency of electrolysis, one needs 16.74 µmol O₂ to deliver about 375 µL at ambient conditions. This should be a good estimate to deliver 250 µL at the pressure range (70-110 kPa) with efficiency losses. The table below shows the area of MnO₂ needed at different coatings.

| Load amount mg/cm² | MnO₂ amount (mmol/cm²) | Area needed (cm²) | Radius needed (mm) | Thickness with Ni layer (mm) |
|---|---|---|---|---|
| 4.2 | 0.048 | 0.693 | 4.7 | 0.16 |
| 8.6 | 0.099 | 0.338 | 3.3 | 0.23-0.28 |
| 15 | 0.173 | 0.194 | 2.5 | 0.34-0.37 |

### 2. pH Effect

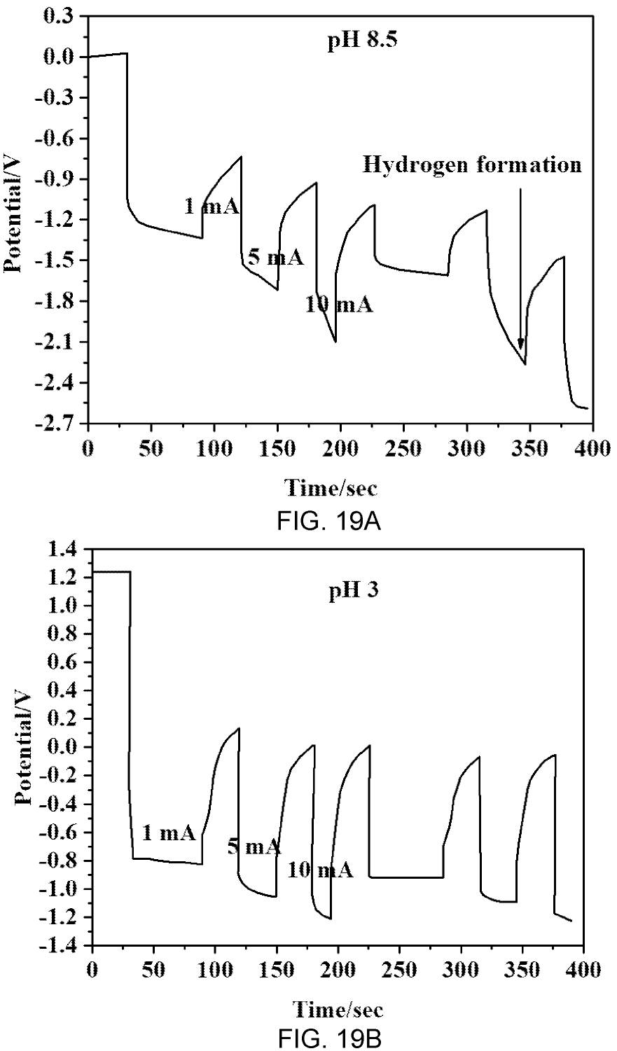

The electrolysis voltage required at three different current levels (1, 5 and 10 mA) with pH 8.5 and pH 3 solutions are shown in FIGS. 19A and 19B, respectively. FIGS. 19A-19B show multistep current mode to monitor the electrolysis voltage at 1, 5 and 10 mA with two pH levels. The voltage over 2 V was generally considered to have hydrogen generation. Under high pH conditions, the voltage to drive 1 mA electrolysis was higher than 1.2 V and that of 10 mA was more than 2.1 V. The hydrogen generation quickly picked up and there was no hydrogen-free pumping with the time. In contrast, under pH 3 conditions, the electrolysis voltage for 1 mA was less than 1 V and that of 10 mA is less than 1.4 V. It was virtually hydrogen-free electrolysis. This result matches the trend deduced herein from the equilibrium potentials. For later tests in this Example, pH 3 phosphate buffer was used.

### 3. Typical Electrolysis With MnO₂ Electrode

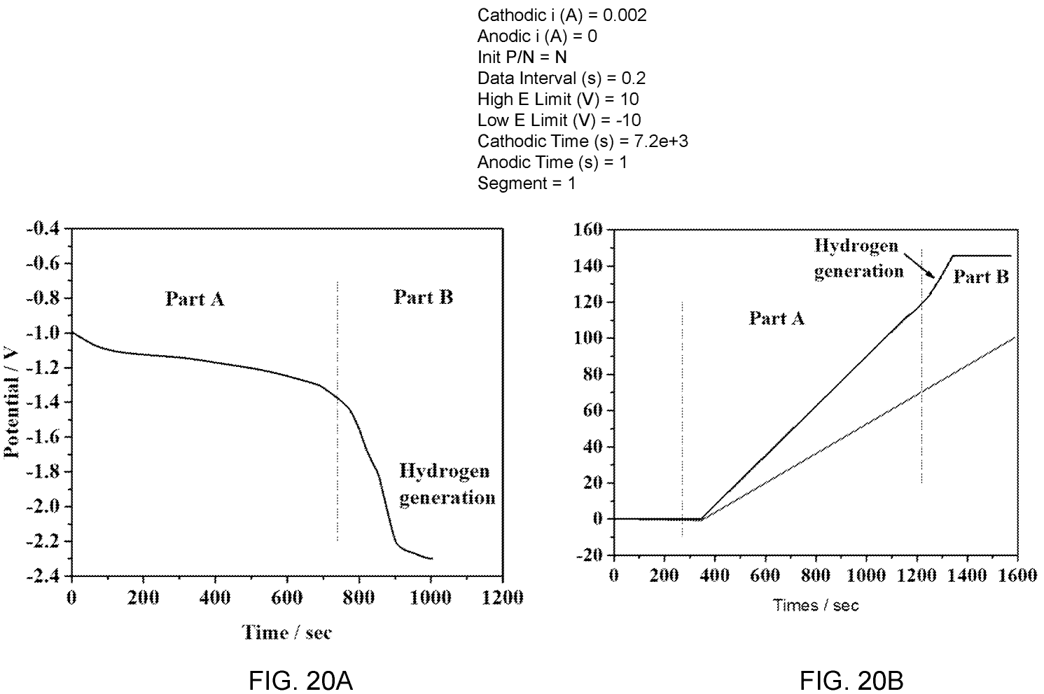

One important question considered was how much MnO₂ was needed to generate enough gases for delivery (250 µL ambient, for example). With the consumption of MnO₂, the actual voltage needed for O₂-only electrolysis was expected to increase (from the Nernst Equation) and eventually the hydrogen generation was expected to start at high enough potentials. There was a transition range when O₂, MnO₂ and H₂ were all involved in electrolysis. FIGS. 20A-20B show the typical electrolysis process with 5×6 mm² 8.6 mg/cm² MnO₂ loaded electrodes and with pH 3 1 M phosphate buffer. FIG. 20A side shows the voltage change with 2 mA electrolysis current, and FIG. 20B shows the reading of a mass balance. It should be noted that the time axes in these plots are offset.

FIGS. 20A-20B shows two stages of electrolysis. The first stage is the long and slow decreasing Part A when the electrolysis voltage is less than 1.4 V. The delivery rate is also stable with 0.126 µL/s, which is very close to the theoretical oxygen-only rate of 0.116 µL/s. The second stage is Part B with drastic changes of electrolysis voltage (quickly from 1.4 V to 2.3 V) and the delivery rate was increased to 0.23 µL/s, while the theoretical water electrolysis rate is 0.347 µL/s. This is believed to be the transition range when all reactions are occurring the same time. For hydrogen-free applications, it is desirable that operation remain in Part A in FIGS. 20A-20B. The result in FIGS. 20A-20B shows the effectiveness of MnO₂ for hydrogen free electrolysis in some embodiments.

### 4. Reliability of Performance

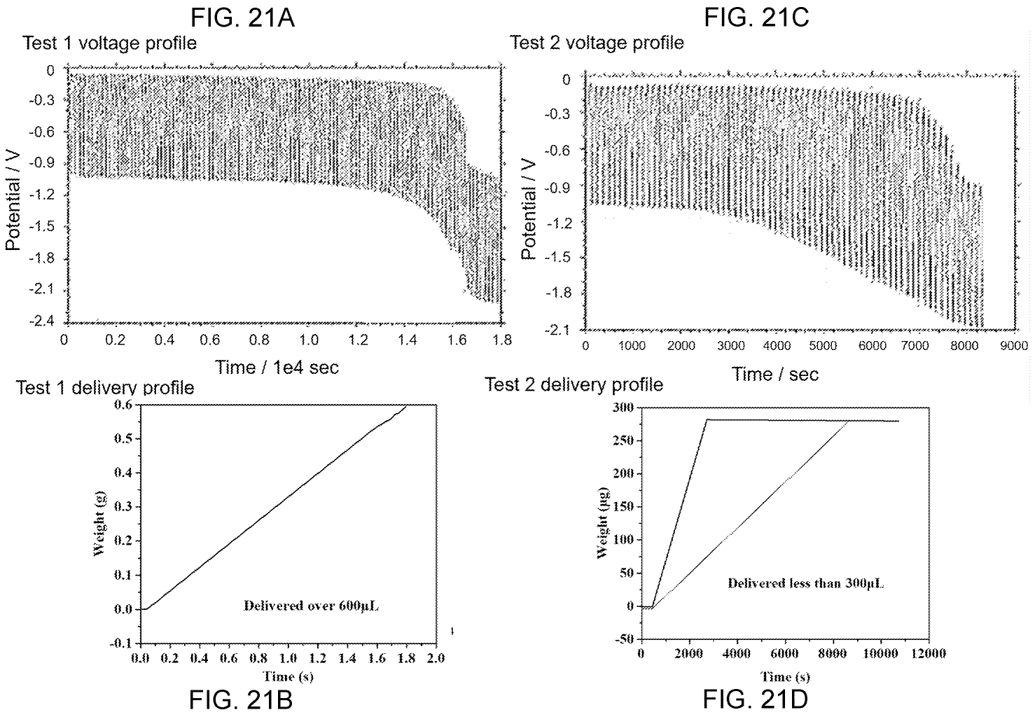

Visual inspection of the MnO₂ coated Ni electrode showed some color variations as well as thickness variations, which may be expected to affect the actual performance. The same tests were performed with two 15 mg/cm² MnO₂ electrodes cut from different region, as shown in FIGS. 21A-21D. FIGS. 21A-21D show electrolysis with 5×6 mm² 15 mg/cm² loaded MnO₂ electrodes with pH 3 1 M phosphate buffer. Two pieces of electrodes were cut from different regions. The results for a first sample are shown in FIGS. 21A-21B and left demonstrated a much longer hydrogen-free pumping regime in time. It was determined to be reasonable to set the hydrogen-free pumping range to be less than 2 V electrolysis voltage. The sample for FIGS. 21A-21B performed more than 4.4 hours hydrogen-free with over 600 µL delivery. The results for a second sample are shown in FIGS. 21C-21D, and that sample only pumped a little over 2 hours with less than 300 µL delivery. This result showed that variation in coatings can affect reactivity, and a uniform coating may contribute to more stable results.

### 5. Recombination After Electrolysis

Recombination of the electrolysis reaction products can be a concern for electrochemical systems such as that of this Example because the electrode potentials are close for O₂ and MnO₂. However, it is believed that the dominating factor is the kinetics, which is slow for turning O₂ back to water.

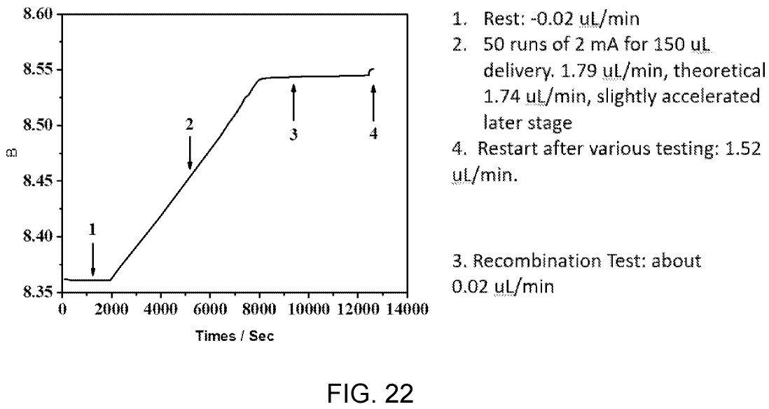

FIG. 22 shows a test result with 15 mg/cm² MnO₂. FIG. 22 shows 150 µL delivery at 2 mA electrolysis with 15 mg/cm² MnO₂ electrode. Four sections are: 1: baseline shift; 2: delivery at 2 mA; 3: Resting for recombination; 4: Restarting pumping. The observed delivery rate of 1.79 µL/min was very close to theoretical hydrogen-free pumping of 1.74 µL/min. The baseline reading dropping at -0.02 µL/min is attributed to the wetting of the electrodes. In the recombination section 3 in FIG. 22 (> 1 h), no clear back flow from recombination was observed. Thus the overall recombination for this system was determined to be acceptable.

### 6. Short Term Stability

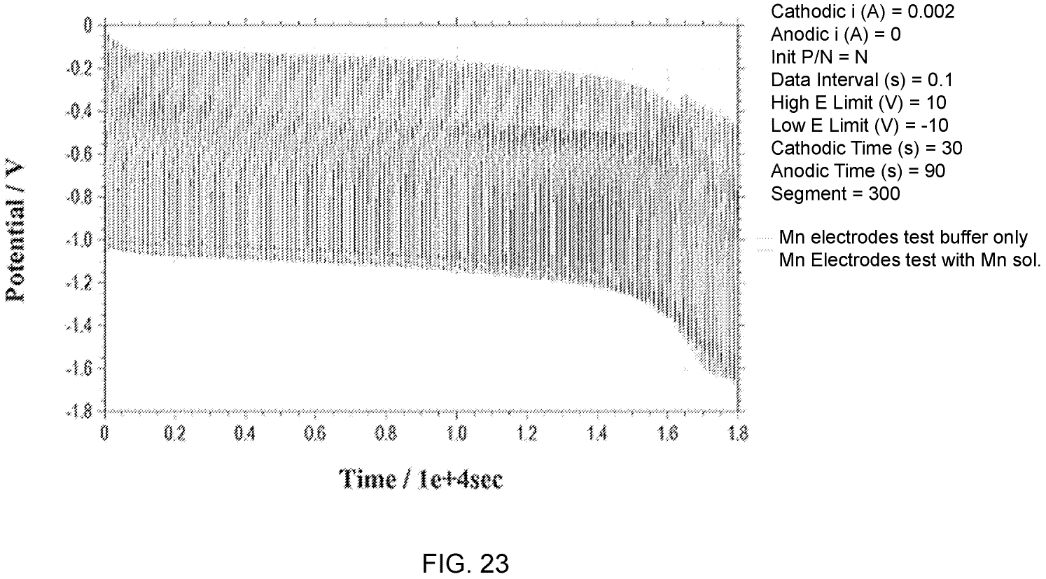

Stability is another consideration because the coating of MnO₂ was considered to be potentially fragile and easy to drop off the electrode. However, as shown in FIG. 23, overnight soaked electrodes performed similarly as the result in FIG. 21 left. FIG. 23 shows results for a ~ 5×6 mm 16 mg/cm² MnO₂ electrode soaked overnight in regular 1 M pH 3 buffer and 1 M pH 3 buffer with saturated Mn²⁺. 2 mA electrolysis was performed, and the voltage curves were observed to overlap. This shows acceptable stability for short term storage of MnO₂ electrodes in the buffer.

### 7. Result With Single Sided Electrodes

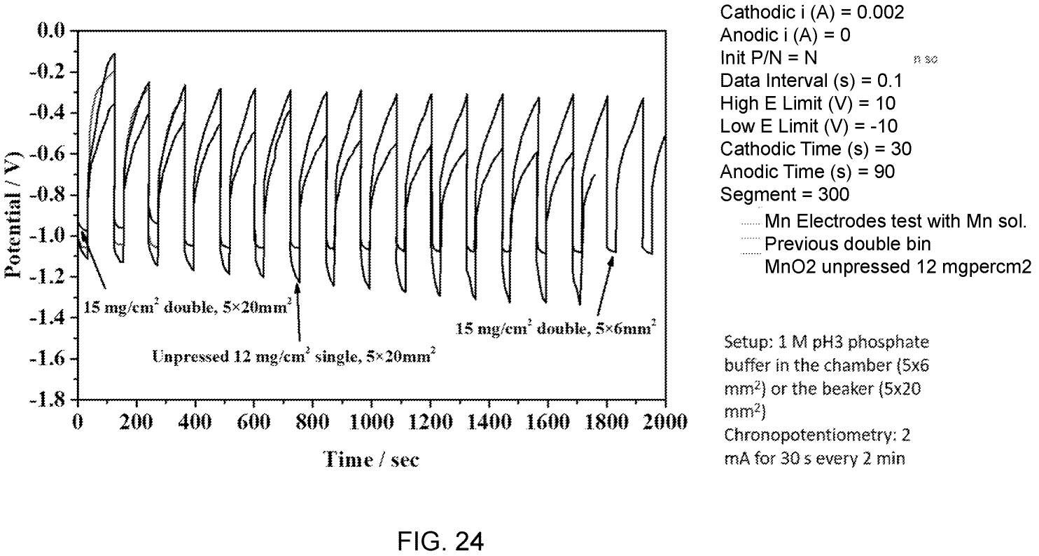

A second batch of electrodes were single-sided with MnO₂ coated on stainless steel (SS). However, only the 5 and 12 mg/cm² coating samples were relatively uniform, and thicker coatings were observed to have had many cracks and easy to fall apart. Direct comparison of these electrodes with the first batch of double-sided electrodes are shown in FIG. 24. The single sided unpressed 12 mg/cm² 5×20 mm² electrode had an initial electrolysis voltage around 1.1 V, but quickly rose to be over 1.4 V and kept rising.

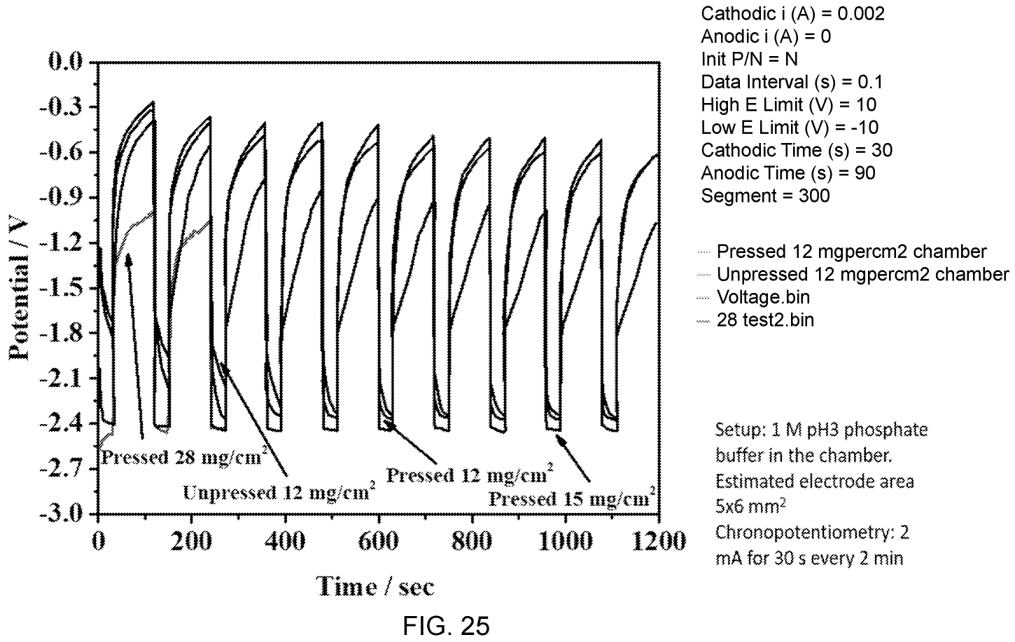

FIG. 25 compares the performance of different single sided MnO₂ coatings in closed chambers with 5×6 mm² with 2 mA electrolysis. None of these electrodes were observed to have performance comparable to the double sided versions because the voltage required for 2 mA electrolysis was observed to quickly rise to be over 2 V. Among them, all the heavy loaded ones were observed to have cracks and lose connection between the MnO₂ layer and SS backing. An unpressed 12 mg/cm² sample was observed to perform slightly better than pressed version, which indicates that certain porosity may help with increasing the overall area for electrolysis and the diffusion limitation may be reduced.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An electrochemical system, comprising:
a first electrode comprising an intercalation compound; and
a second electrode having an opposite polarity of the first electrode;
wherein the electrochemical system is configured such that application of at least one magnitude of voltage across the first electrode and second electrode:
causes the intercalation compound in the first electrode to undergo an intercalation reaction, and
generates a gaseous species at the second electrode via a gas generation reaction; and
wherein, under at least one condition, an equilibrium potential difference between the intercalation reaction and the gas generation reaction is less than or equal to 2 V.

2. An electrochemical system, comprising:
a first electrode comprising an intercalation compound; and
a second electrode having an opposite polarity of the first electrode;
wherein the electrochemical system is configured such that application of a first magnitude of voltage across the first electrode and second electrode:
causes the intercalation compound in the first electrode to undergo an intercalation reaction, and
generates a gaseous species at the second electrode via a gas generation reaction;
wherein the electrochemical system is configured such that application of a second magnitude of voltage across the first electrode and second electrode:
causes the intercalation compound in the first electrode to undergo a deintercalation reaction, and
consumes the gaseous species at the second electrode via a gas consumption reaction; and
wherein the electrochemical system is configured such that when no electrical current is passed between the first electrode and the second electrode, the gas consumption reaction does not occur at the first electrode or the gas consumption reaction occurs at a rate of less than or equal to 5 mol% per day.

3. An electrochemical system, comprising:
a first electrode comprising an intercalation compound; and
a second electrode having an opposite polarity of the first electrode;
wherein the electrochemical system is configured such that application of at least one magnitude of voltage across the first electrode and second electrode:
causes the intercalation compound in the first electrode to undergo an intercalation reaction, and
generates a gaseous product at the second electrode via a gas generation reaction; and
wherein the intercalation compound can undergo an intercalation reaction upon a first use of the electrochemical system.

4. An electrochemical system, comprising:
a chamber comprising a compliant surface at least partially enclosing an interior volume of the chamber;
a first electrode exposed to the interior volume of the chamber and having a polarity, the first electrode comprising an electroactive compound comprising manganese or iron; and
a second electrode exposed to the interior volume of the chamber and having an opposite polarity of the first electrode;
wherein the electrochemical system is configured such that application of at least one magnitude of voltage across the first electrode and second electrode:
causes at least some of the manganese or iron to undergo a change in oxidation state; and
generates a gaseous species at the second electrode at a pressure sufficient to deform the compliant surface.

5. A method, comprising:
in an electrochemical cell comprising:
a chamber comprising a compliant surface;
a first electrode; and
a second electrode in the chamber having an opposite polarity of the first electrode:
applying a voltage having a magnitude of less than or equal to 3 V across the across the first electrode and second electrode such that a gaseous species is generated at the second electrode.

6. A method, comprising
in an electrochemical cell comprising:
a chamber comprising a compliant surface;
a first electrode; and
a second electrode in the chamber having an opposite polarity of the first electrode:
applying a voltage across the first electrode and second electrode such that:
oxygen gas is generated at the second electrode; and
greater than or equal to 80 mole percent of a total amount of gas generated in the chamber during the applying step is oxygen gas; and
deforming the compliant surface using the generated gas.

7. A method, comprising:
in an electrochemical cell comprising:
a chamber;
a first electrode in the chamber having a polarity; and
a second electrode in the chamber having an opposite polarity of the first electrode:
passing a first current through the first electrode and second electrode for a first period of time such that a gaseous species is generated;
deforming the compliant surface using the generated gaseous species;
passing a second current through the first electrode and second electrode for a second period of time such that a portion of the gaseous species is consumed; and
determining an amount of the gaseous species consumed during the passing of the second current.

8. The electrochemical system or method of any preceding claim, wherein the electrochemical system comprises a chamber and the first electrode and second electrode are each in the chamber.

9. The electrochemical system or method of any preceding claim, wherein the chamber comprises a compliant surface.

10. The electrochemical system or method of any preceding claim, wherein the compliant surface is a membrane.

11. The electrochemical system or method of any preceding claim, wherein the application of at least one magnitude of voltage generates the gaseous species at the second electrode at a pressure sufficient to deform the compliant surface.

12. The electrochemical system or method of any preceding claim, wherein the compliant surface at least partially encloses an interior volume of the chamber.

13. The electrochemical system or method of any preceding claim, wherein the first electrode and/or the second electrode is exposed to an interior volume of the chamber.

14. The electrochemical system or method of any preceding claim, wherein, under at least one condition, an equilibrium potential difference between the gas generation reaction and the intercalation reaction is less than or equal to 1 V, and greater than or equal to -1 V.

15. The electrochemical system or method of any preceding claim, wherein, in an aqueous solution having a pH from 6-16, an equilibrium potential difference between the gas generation reaction and the intercalation reaction is less than or equal to 2 V.

16. The electrochemical system or method of any preceding claim, wherein, the intercalation compound can reversibly undergo the intercalation reaction and a deintercalation reaction.

17. The electrochemical system or method of any preceding claim, wherein the first electrode comprises an intercalation compound.

18. The electrochemical system or method of any preceding claim, wherein the intercalation compound can undergo an intercalation reaction upon a first use of the electrochemical system.

19. The electrochemical system or method of any preceding claim, wherein the intercalation compound is a metal ion/proton intercalation compound.

20. The electrochemical system or method of any preceding claim, wherein the intercalation compound is a lithium ion intercalation compound.

21. The electrochemical system or method of any preceding claim, wherein the intercalation compound comprises a transition-metal oxide.

22. The electrochemical system or method of any preceding claim, wherein the transition metal oxide comprises a manganese oxide.

23. The electrochemical system or method of any preceding claim, wherein the intercalation compound comprises a transition-metal oxyanion.

24. The electrochemical system or method of any preceding claim, wherein the transition metal oxyanion comprises an iron phosphate.

25. The electrochemical system or method of any preceding claim, wherein the intercalation compound is a lithium ion intercalation compound.

26. The electrochemical system or method of any preceding claim, wherein the intercalation compound comprises a lithium manganese oxide (LiMnO₂, LiMn₂O₄, or Li₄Mn₅O₁₂).

27. The electrochemical system or method of any preceding claim, wherein the intercalation compound comprises lithium iron phosphate (LiFePO₄).

28. The electrochemical system or method of any preceding claim, wherein, the lithium manganese oxide can be cycled in an alkaline electrolyte.

29. The electrochemical system or method of any preceding claim, wherein, the alkaline electrolyte comprises one or more additives.

30. The electrochemical system or method of any preceding claim, wherein the one or more additives decreases an equilibrium potential of a hydrogen evolution reaction compared to that of an otherwise identical electrolyte lacking the one or more additives.

31. The electrochemical system or method of any preceding claim, further comprising a surface layer at least partially coating the first electrode, the surface layer configured to decrease side reactions.

32. The electrochemical system or method of any preceding claim, wherein the second electrode comprises a three dimensional porous current collector.

33. The electrochemical system or method of any preceding claim, wherein the second electrode comprises one or more catalysts for an oxygen evolution reaction and/or an oxygen reduction reaction.

34. The electrochemical system or method of any preceding claim, wherein the three dimensional porous current collector comprises open-channel pores with largest cross-sectional dimensions from 10 nm to 1 mm.

35. The electrochemical system or method of any preceding claim, wherein the electroactive compound comprises manganese or iron.

36. The electrochemical system or method of any preceding claim, wherein the electroactive compound comprising manganese comprises a manganese oxide.

37. The electrochemical system or method of any preceding claim, wherein the manganese oxide comprises manganese (IV) oxide (MnO₂).

38. The electrochemical system or method of any preceding claim, wherein the electroactive compound is air-stable.

39. The electrochemical system or method of any preceding claim, wherein the interior volume of the chamber can be at least partially filled with an electrolyte solution.

40. The electrochemical system or method of any preceding claim, wherein the gaseous species is oxygen gas (O₂).

41. The electrochemical system or method of any preceding claim, wherein the oxygen gas is generated via the oxidation of water (H₂O).

42. The electrochemical system or method of any preceding claim, wherein the first electrode comprises a substrate and the electroactive compound on at least a portion of the substrate.

43. The electrochemical system or method of any preceding claim, wherein the electroactive compound is part of a composite layer.

44. The electrochemical system or method of any preceding claim, wherein the second electrode comprises a non-platinum substrate.

45. The electrochemical system or method of any preceding claim, wherein the substrate of the second electrode is at least partially coated with an oxygen-reduction catalyst.

46. The electrochemical system or method of any preceding claim, wherein the substrate of the first electrode comprises an iron alloy.

47. The electrochemical system or method of any preceding claim, wherein the first electrode and/or the second electrode is at least partially coated with a polymer electrolyte.

48. The electrochemical system or method of any preceding claim, wherein the second electrode is configured to generate bubbles of the gaseous species.

49. The electrochemical system or method of any preceding claim, wherein the chamber has a volume of less than or equal to 50 mL.

50. The electrochemical system or method of any preceding claim, wherein the at least one magnitude of voltage is less than or equal to 3 V.

51. The electrochemical system or method of any preceding claim, wherein the pressure sufficient to deform the compliant surface is less than or equal to 30 kPa.

52. The method of any preceding claim, wherein the method further comprises deforming the compliant surface using the generated gaseous species.

53. The method of any preceding claim, wherein a magnitude of the applied voltage is less than or equal to 3 V, less than or equal to 2.8 V, less than or equal to 2.6 V, less than or equal to 2.4 V, less than or equal to 2.4 V, less than or equal to 2.2 V, less than or equal to 2.0 V, less than or equal to 1.8 V, less than or equal to 1.6 V, less than or equal to 1.4 V, less than or equal to 1.3 V, less than or equal to 1.2 V, less than or equal to 1.1 V, less than or equal to 1.0 V, less than or equal to 0.9 V, or less than or equal to 0.8 V.

54. The method of any preceding claim, wherein deforming the compliant surface causes a fluid to flow at least partially through a channel.

55. The method of any preceding claim, wherein the channel is in fluid communication with the compliant surface.

56. The method of any preceding claim, wherein greater than or equal to 80 mol%, greater than or equal to 85 mol%, greater than or equal to 90 mol%, greater than or equal to 95 mol%, greater than or equal to 98 mol%, greater than or equal to 99 mol%, or greater than or equal to 99 mol% of a total amount of gaseous species generated in the chamber during the applying step is oxygen gas.

57. The method of any preceding claim, wherein the method further comprises applying a second voltage across the first electrode and the second electrode such that at least a portion of the gaseous species is consumed in an electrochemical reaction.

58. The method of any preceding claim, wherein the at least a portion of the gaseous species is consumed at the second electrode.

59. The method of any preceding claim, wherein the method further comprises determining an amount of gaseous species consumed in the electrochemical cell using at least one electrochemical measurement.

60. The method of any preceding claim, wherein a volume of gaseous species generated is greater than or equal to 5 µL and less than or equal to 5 mL.

61. The method of any preceding claim, wherein during the step of applying the second voltage or passing the second electrical current, at least some of an intercalated metal ion or proton is released from the intercalation compound.

62. The method of any preceding claim, wherein the chamber comprises an electrolyte in contact with the first electrode and/or the second electrode.

63. The method of any preceding claim, wherein the electrolyte has a pH of greater than or equal to 0 and less than or equal to 16.
